(19) United States
(12) Patent Application Publication
(10) Pub. No.: US 2025/0360306 A1
(43) Pub. Date: Nov. 27, 2025

(54) FLEXIBLE LAYER FOR USE IN A TUMOR TREATING FIELDS TRANSDUCER

(71) Applicant: Novocure GmbH, Baar (CH)

(72) Inventors: Elie YAACOBI, Haifa (IL); Noa HALAVEE, Tel Aviv (IL); Dmitry GOLOM, Haifa (IL)

(73) Assignee: Novocure GmbH, Baar (CH)

(21) Appl. No.: 19/290,191

(22) Filed: Aug. 4, 2025

Related U.S. Application Data
(63) Continuation-in-part of application No. 18/611,390, filed on Mar. 20, 2024.
Application No. 18/611,390 is a continuation-in-part of application No. 18/216,151, filed on Jun. 29, 2023.
(60) Provisional application No. 63/357,278, filed on Jun. 30, 2022.
(60) Provisional application No. 63/357,390, filed on Jun. 30, 2022.
(60) Provisional application No. 63/420,950, filed on Oct. 31, 2022.
(60) Provisional application No. 63/421,005, filed on Oct. 31, 2022.
(60) Provisional application No. 63/679,495, filed on Aug. 5, 2024.
(60) Provisional application No. 63/701,217, filed on Sep. 30, 2024.
(60) Provisional application No. 63/740,589, filed on Dec. 31, 2024.

(51) Int. Cl. A61N 1/04; A61N 1/36

(52) U.S. Cl. CPC A61N 1/0496; A61N 1/0476; A61N 1/36002

(57) ABSTRACT

A transducer apparatus for delivering tumor treating fields to a subject's body, the transducer apparatus comprising an array of one or more electrode elements, the array configured to be positioned over the subject's body with a front face of the array facing the subject's body, an anisotropic material layer electrically coupled to the array and located on a front side of the front face of the array, the anisotropic material layer comprising a front face and a back face, the back face facing the array, and a flexible layer coupled to the front face of the anisotropic material layer and configured to contact the subject's body, wherein the flexible layer comprises a flexible material and, optionally, an adhesive.

### CROSS-REFERENCE TO RELATED APPLICATIONS

This application claims the benefit of and priority to U.S. Provisional Patent Application No. 63/740,589, filed Dec. 31, 2024; U.S. Provisional Patent Application No. 63/357,278, filed Jun. 30, 2022; U.S. Provisional Patent Application No. 63/357,390, filed Jun. 30, 2022; U.S. Provisional Patent Application No. 63/420,950, filed Oct. 31, 2022; U.S. Provisional Patent Application No. 63/421,005, filed Oct. 31, 2022; U.S. patent application Ser. No. 18/216,151, filed Jun. 29, 2023; and U.S. patent application Ser. No. 18/611,390, filed Mar. 20, 2024, the contents of each of which are all incorporated herein by reference in their entireties. This application further claims the benefit of and priority to U.S. Provisional Patent Application No. 63/679,495, filed Aug. 5, 2024, the contents of which is incorporated herein by reference in its entirety. This application further claims the benefit of and priority to U.S. Provisional Patent Application No. 63/701,217, filed Sep. 30, 2024, the contents of which is incorporated herein by reference in its entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. In current commercial systems, TTFields are induced non-invasively into a region of interest by electrode assemblies (also known as electrode arrays, transducer arrays, or transducers) placed on the patient's body and applying alternating current (AC) voltages between the transducers. Conventionally, one or more pairs of transducers (e.g., a first pair of transducers and a second pair of transducers) are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIGS. 1A-1H depict example transducers with a non-conductive border according to some embodiments.

FIGS. 2A-2E depict example transducers with a non-conductive border according to some embodiments.

FIGS. 3A and 3B depict a top plan view and a bottom plan view, respectively, of an example transducer apparatus according to some embodiments.

FIG. 3C depicts a cross-section view of the example transducer apparatus of FIGS. 3A and 3B taken across the section 3C-3C′ according to some embodiments.

FIGS. 4A and 4B depict a top plan view (i.e., a back face plan view) and a bottom plan view (i.e., a front face view), respectively, of an example transducer apparatus according to some embodiments.

FIG. 4C depicts a cross-section view of the example transducer apparatus of FIGS. 4A and 4B taken across section 4C-4C′ according to some embodiments.

FIG. 4D depicts a cross-section view of the example transducer apparatus of FIGS. 4A and 4B taken across section 4D-4D′ according to some embodiments.

FIG. 4E depicts a cross-section view of an exemplary transducer apparatus according to some embodiments.

FIGS. 5A to 5C depict a bottom plan view, a right plan view, and a top plan view, respectively, of an example transducer apparatus according to some embodiments.

FIG. 6 depicts an exploded view of an example transducer apparatus according to some embodiments.

FIGS. 7A and 7B depict a bottom plan view and a top plan view, respectively, of an example transducer apparatus according to some embodiments.

Various embodiments are described in detail below with reference to the accompanying drawings, wherein like reference numerals represent like elements, and wherein descriptions of like elements may not be repeated for every embodiment, but may be considered to be the same if previously described herein.

The figures provided herein are for illustrative purposes and may not be to scale. Variations in dimensions, proportions, and configurations may exist between the figures and the actual embodiments. The figures are intended to facilitate understanding of the embodiments and should not be construed as limiting the scope of the disclosure.

### DESCRIPTION OF EMBODIMENTS

This application describes exemplary transducer apparatuses used to apply TTFields to a subject's body, for example, for treating one or more cancers. This application also describes exemplary methods to apply TTFields to a subject's body using transducers.

TTFields may include low-intensity (e.g., about 1 V/cm to about 10 V/cm) alternating electric fields of medium frequencies (e.g., about 50 kHz to about 1 MHz, and, for some embodiments, about 100 kHz to about 300 kHz) that when applied to a conductive medium, such as a human body, via electrodes may be used, for example, to treat tumors as described in U.S. Pat. Nos. 7,016,725, 7,089,054, 7,333,852, 7,565,205, 7,805,201, and 8,244,345 by Palti and in Eilon D. Kirson et al., “Disruption of Cancer Cell Replication by Alternating Electric Fields,” Cancer Res. 2004 64:3288-3295; all of which are hereby incorporated by reference in their entirety.

Transducers used to apply TTFields to a subject's body often include one or more electrode elements electrically coupled together on a substrate and attached to the subject's body at a desired location, for example, via an adhesive backing of the substrate or a separately applied adhesive. Proper positioning of the transducer apparatus over a target region (e.g., a tumor) can affect performance of treatment. However, proper placement can be difficult, depending on where the transducer apparatus is being placed and particularly when the subject is placing the transducer apparatus without assistance or even with the assistance of a caregiver. Improper positioning may lead to diminished effectiveness. Further, positioning of the transducer apparatus over certain placement areas (e.g. on a subject's sternum, back, and oblique/latissimus dorsi area) may be difficult due to the contour of the areas and bi-direction tension that results in a risk of detachment of the transducer apparatus when the subject shifts or moves. Positioning over certain placement areas (e.g. a subject's sternum) is also difficult due to sweating of the subject, which may also result in a risk of detachment. Accordingly, difficulties in positioning may lead to a decrease in the performance of the treatment. Accordingly, a way to assist with properly positioning transducers may be desirable.

As recognized by the inventors, a transducer apparatus including an anisotropic material layer may cause irritation to the subject due to the rigidness of the anisotropic material layer. For example, the anisotropic material layer may be relatively inflexible, like a sturdy piece of cardboard, and as such, may not easily conform to a subject's body, which is typically non-planar over a large area or which may bend and change shape as the subject moves. Moreover, due to a non-planar surface on a subject and/or a surface of the subject bending and changing shape, the anisotropic material layer may deform or even crack, resulting in the transducer array producing a less than desired electric field. The inventors have discovered that using a hole in, or substantially in, the center or the middle of the anisotropic material layer may help to alleviate this problem. The inventors have also discovered that using one or more slits extending from a point on the outer perimeter of the anisotropic material layer towards the interior of the anisotropic material layer may also help to alleviate this problem. Further, the inventors have discovered that the use of a flexible layer, such as foam, disposed between the anisotropic material layer and the subject's body may also help to alleviate this problem.

As further recognized by the inventors, a transducer apparatus including an anisotropic material layer may cause irritation to the subject due to the sharp edges of the anisotropic material layer. When a piece of anisotropic material is cut to size to be used as an anisotropic material layer in a transducer apparatus, the edges of the transducer apparatus may be sharp. As such, when these edges come in contact with a subject, the subject may experience discomfort. To reduce this discomfort from the transducer apparatus, the transducer apparatus may be placed in a different, non-desired position on the subject for delivery of TTFields, which may result in the subject receiving less than a desired dosage of TTFields. Further, to reduce this discomfort from the transducer apparatus, the subject may use the transducer apparatus less, which may result in the subject receiving less than a desired dosage of TTFields. The inventors have discovered that using a foam layer with the anisotropic material layer may help to alleviate this discomfort experienced by a subject. The inventors have further discovered that the foam layer may advantageously provide some additional flexibility for the array when the subject moves or twists the body part associated with the location of the array. Further, the added flexibility is enhanced when the foam layer covers (on the skin-facing side) a slit or central hole in the anisotropic material layer.

This application describes exemplary transducer apparatuses used to apply TTFields to a subject's body for treating one or more cancers. Transducers used to apply TTFields to a subject's body may include one or more electrode elements coupled together on a substrate and attached to the subject's body at a desired location, for example, via an adhesive layer on the substrate or a separately applied adhesive. Transducers may include one or more conductive material layers located between the electrode elements and the subject's body upon attachment of the transducer to the subject's body. Such conductive material layers may include, for example, a conductive skin-contact layer such as a hydrogel or a conductive adhesive layer located against the subject's body. The conductive adhesive layer may take the form of an adhesive matrix material having conductive particles (e.g., carbon fibers or carbon black powder) embedded at least partially in the adhesive matrix material. Additionally, the conductive material layer(s) may include a conductive anisotropic material layer taking the form of a carbon layer, a graphite layer, or others. The conductive anisotropic material layer may have different thermal and/or electrical conductivities in a direction perpendicular to a face of the transducer (z-direction) than in directions parallel to the transducer face (directions in the x-y plane). Conductive material layer(s) having greater thermal conductivity in the x-y plane than in the z-direction can spread out heat generated by the electrode elements within an x-y plane while conducting electricity from the electrode elements in a z-direction toward the subject's body. This allows greater currents to be applied to the electrode elements while maintaining the temperature at the subject's skin under a maximum operating temperature.

Descriptions of embodiments related to specific exemplary drawings herein may be applicable, and may be combined with, descriptions of embodiments related to other exemplary drawings herein unless otherwise indicated herein or otherwise clearly contradicted by context.

### Example First Transducers

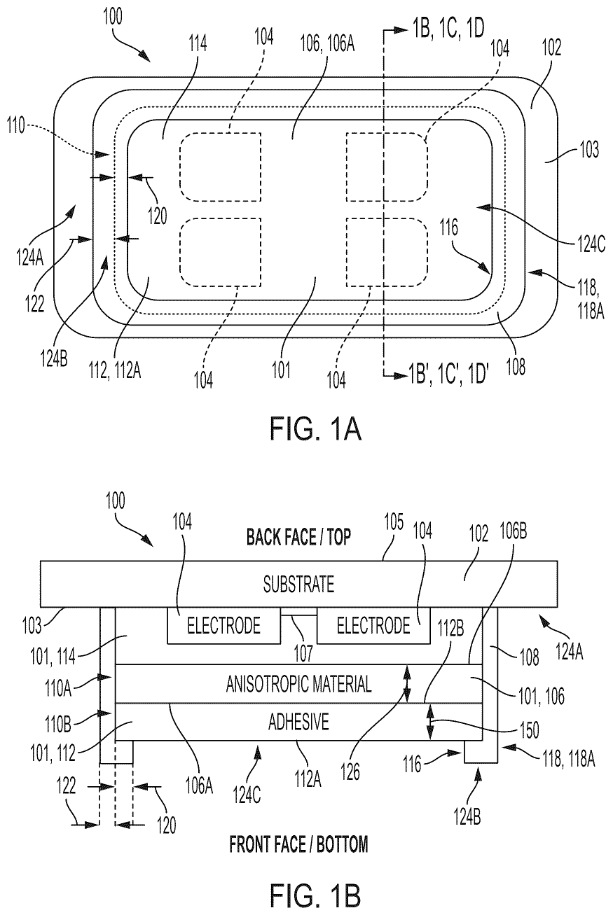

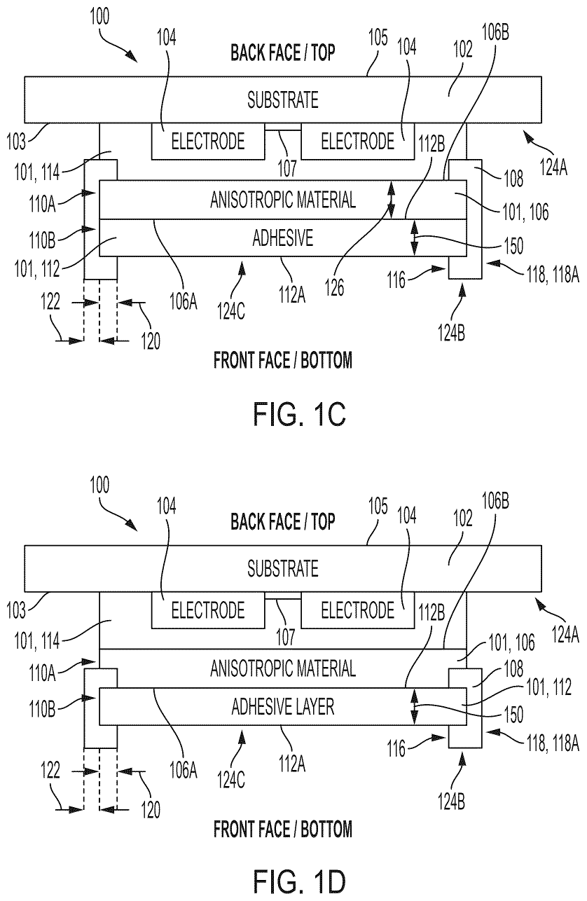

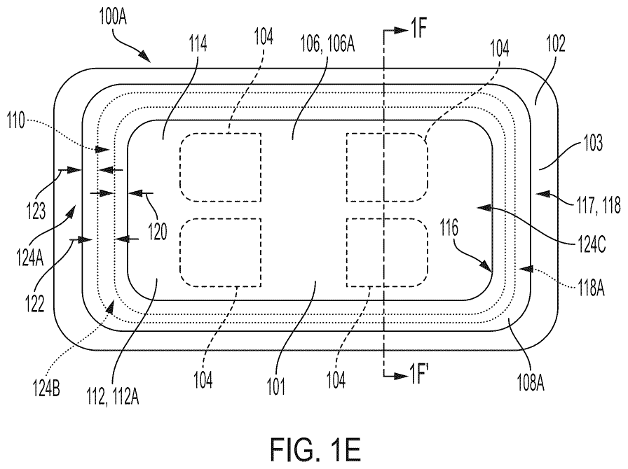

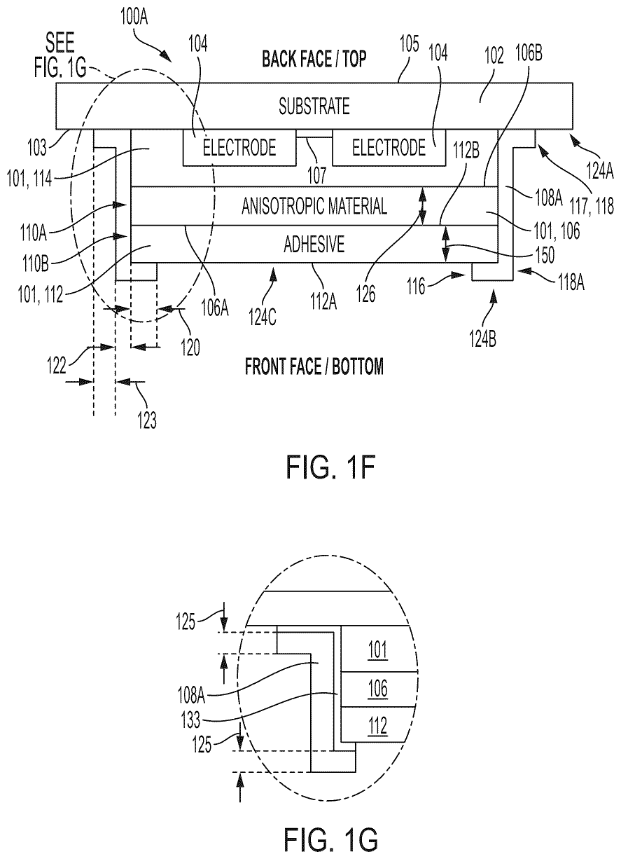

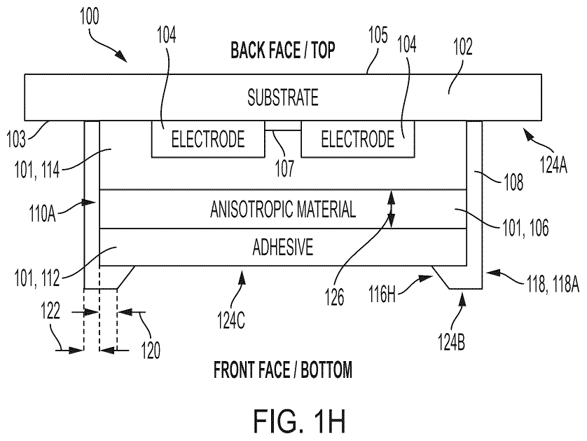

FIG. 1A depicts a bottom view (i.e., a front face view, or a skin-facing view) of an example transducer apparatus 100 according to some embodiments. FIGS. 1B, 1C, and 1D depict example cross-section views of the example transducer apparatus 100 of FIG. 1A taken across sections 1B-1B′, 1C-1C′, and 1D-1D′, respectively, according to some embodiments. FIG. 1H depicts an example alternative cross-section view of the example transducer apparatus 100 of FIG. 1A.

FIG. 1E depicts a bottom view (i.e., a front face view, or a skin-facing view) of an example transducer apparatus 100A according to some embodiments. FIG. 1F depicts a cross-section view of the example transducer apparatus 100A of FIG. 1E taken across section 1F-1F′ according to some embodiments. FIG. 1G depicts a zoom-in view of adhesive area 1G of the example transducer apparatus 100A of FIG. 1F.

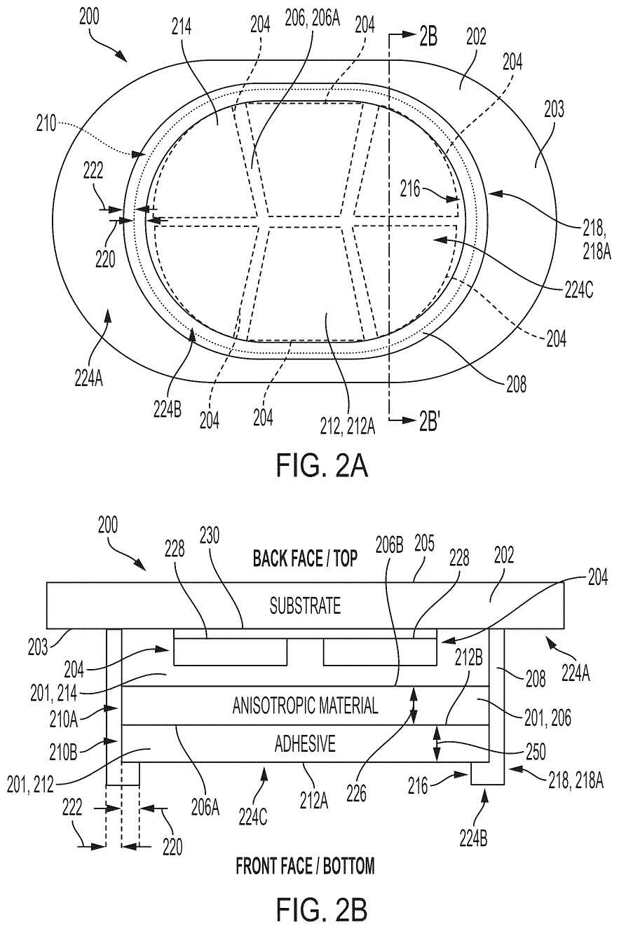

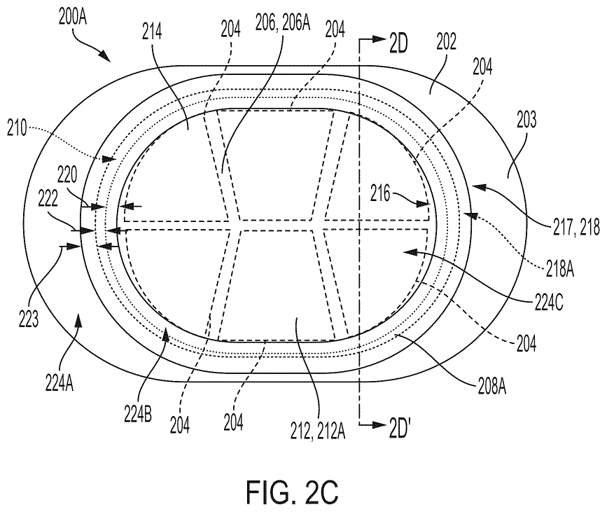

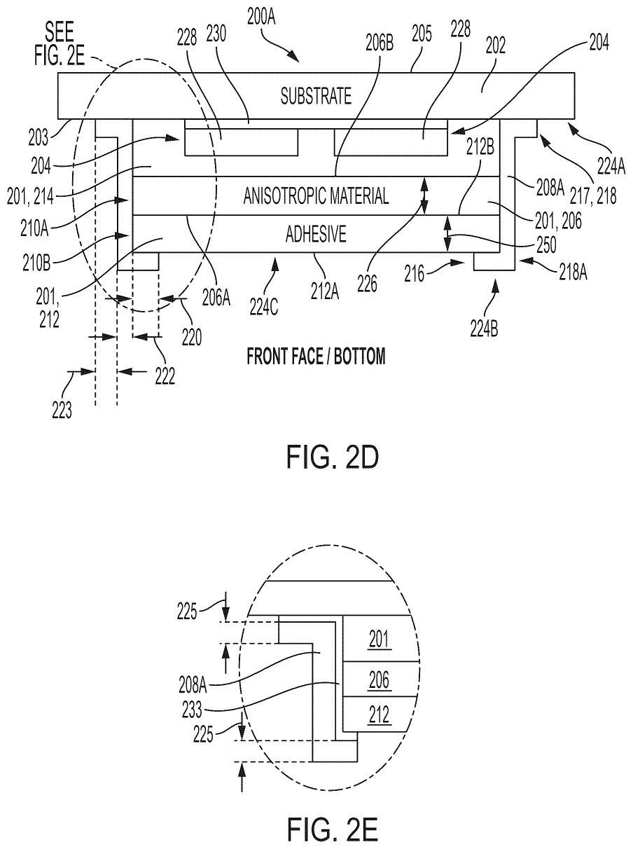

FIG. 2A depicts a bottom view (i.e., a front face view, or a skin-facing view) of an example transducer apparatus 200 according to some embodiments. FIG. 2A depicts a cross-section view of the example transducer apparatus 200 of FIG. 2A taken across section 2B-2B′ according to some embodiments.

FIG. 2C depicts a bottom view (i.e., a front face view, or a skin-facing view) of an example transducer apparatus 200A according to some embodiments. FIG. 2D depicts a cross-section view of the example transducer apparatus 200A of FIG. 2C taken across section 2D-2D′ according to some embodiments. FIG. 2E depicts a zoom-in view of adhesive area 2E of the example transducer apparatus 200A of FIG. 2D.

In FIGS. 1A-2E, the transducer 100, 100A, 200, 200A includes a substrate 102, 202, array of at least one electrode 104, 204 coupled to the substrate 102, 202, and an anisotropic material layer 106, 206 coupled to the array of at least one electrode 104, 204. The substrate 102, 202 has a front face 103, 203 and a back face 105, 205, and the electrode element(s) 104, 204 are located on a side of the front face 103, 203 of the substrate 102, 202. As illustrated, the electrode element(s) 104, 204 are located between the substrate 102, 202 and the anisotropic material layer 106, 206. As shown in FIGS. 1A-2E, the anisotropic material layer 106, 206 has a front face 106A, 206A and a back face 106B, 206B, with the back face facing the electrode element(s) 104, 204

The transducer 100, 100A, 200, 200A of each of FIGS. 1A-2E may be affixed to the subject's body via the substrate 102, 202. Suitable materials for the substrate 102, 202 may include, for example, cloth, foam, flexible plastic, and/or a conductive medical gel or adhesive. The substrate may take the form of an adhesive bandage (e.g., a medical bandage).

In FIGS. 1A-2E, the transducers 100, 100A, 200, 200A comprise arrays of substantially flat electrode element(s) 104, 204. For each figure, the array of electrode elements 104, 204 may be capacitively coupled. In one example, as shown in FIGS. 1B, 1C, 1D, 1F, and 1H the electrode elements 104 may be ceramic electrode elements coupled to each other via conductive wiring 107. When viewed in a direction perpendicular to its face, the ceramic electrode elements may be circular shaped or non-circular shaped (e.g., 104 in FIGS. 1A and 1E). In another example, as shown in FIGS. 2B and 2D, the electrode elements 204 may be non-ceramic dielectric materials positioned over a plurality of flat conductors. When viewed in a direction perpendicular to its face, the non-ceramic electrode elements may take any desired shape (e.g., elements 204 in FIGS. 2A and 2C). Examples of non-ceramic dielectric materials positioned over flat conductors may include polymer films 228 disposed over pads on a printed circuit board 230 or over substantially planar pieces of metal. Preferably, such polymer films may have a high dielectric constant, for example having a dielectric constant greater than 10. In other embodiments, the array of electrode elements 104, 204 may not be capacitively coupled, and there may not be dielectric material associated with the electrode elements 104, 204. The electrode elements 104, 204 may take any of these forms without departing from the scope of the present disclosure.

The transducer 100, 100A, 200, 200A may also include at least one conductive material layer 101, 201. In some embodiments, the conductive material layer 101, 201 may be an anisotropic material layer 106, 206 coupled to the array of at least one electrode 104, 204. As shown in FIGS. 1A-2E, the electrode element(s) 104, 204 may be located between the substrate 102, 202 and the anisotropic material layer 106, 206. As shown in FIGS. 1A-2E, the anisotropic material layer 106, 206 may have a front face 106A, 206A and a back face 106B, 206B, with the back face facing the electrode element(s) 104, 204. The anisotropic material layer 106, 206 may include any of the features described in further detail below with reference to the anisotropic material layer 310. In some embodiments, the conductive material layer 101, 201 may be a hydrogel layer or an electrically conductive adhesive layer electrically coupled to the array of at least one electrode 104, 204. The hydrogel layer or electrically conductive adhesive layer may be located on an opposite side of the electrode element(s) 104, 204 from the substrate 102, 202. The hydrogel layer or electrically conductive adhesive layer may be a conductive skin contact adhesive layer 112, 212. As shown in FIGS. 1A-2E, the electrically conductive skin contact adhesive layer 112, 212 may include a front face 112A, 212A and a back face 112B, 212B, with the back face facing the electrode element(s) 104, 204. As illustrated, when an anisotropic material layer 106, 206 is present in the transducer 100, 100A, 200, 200A, the anisotropic material layer 106, 206 may be located between the electrode element(s) 104, 204 and the electrically conductive skin contact adhesive layer 112, 212. Alternatively, or additionally, a hydrogel layer or electrically conductive adhesive layer may function as an upper adhesive layer 114, 214 located between the electrode element(s) 104, 204 and the anisotropic material layer 106, 206. In some embodiments, the anisotropic material layer 106, 206 may be sandwiched between two layers of hydrogel, or sandwiched between two layers of electrically conductive adhesive, or sandwiched between one layer of each.

The anisotropic material layer 106, 206 of FIGS. 1A-2E may be any conductive layer having different thermal and/or electrical conductivities in a direction perpendicular to the front face 103, 203 of the substrate 102, 202 than in directions that are parallel to the front face 103, 203. The anisotropic material layer may be anisotropic with respect to electrical conductivity properties, anisotropic with respect to thermal properties, or both. This allows the anisotropic material layer to spread out current and/or heat over a larger surface area. In each case, this lowers the temperature of hot spots and raises the temperature of cooler regions when a given AC voltage is applied to the array of electrode elements. Accordingly, the current may be increased without exceeding a safety temperature threshold at any point on the subject's skin. The anisotropic material layer may be a sheet of graphite, such as a sheet of synthetic graphite. The anisotropic material layer may be a sheet of pyrolytic graphite, graphitized polymer film, a graphite foil made from compressed high purity exfoliated mineral graphite, or some other material. Other details regarding the anisotropic material layer and properties thereof are described in U.S. Patent Application Publication No. 2023/0037806 A1, Wasserman et al., Feb. 9, 2023, which is hereby incorporated by reference in the present disclosure.

The electrically conductive skin contact adhesive layer 112, 212 and/or the electrically conductive upper adhesive layer 114, 214 may be a composite adhesive layer. For example, the electrically conductive adhesive layer 112, 212; or 114, 214 may comprise a plurality of electrically conductive particles embedded at least partially within an adhesive matrix material. The electrically conductive particles may provide enhanced electrical conductivity in the x-y plane of the adhesive layer. The electrically conductive particles may include carbon granules, carbon flakes, graphite powder, carbon black powder, carbon nanoparticles, carbon nanotubes, and the like. The electrically conductive particles may include electrically conductive fibers, such as carbon fibers, or carbon wires or nanowires. The electrically conductive particles may comprise graphite. The plurality of electrically conductive particles may comprise a sheet of fibers embedded in the adhesive matrix material. The sheet of fibers may be in the form of a mesh layer that can be cut to any desired shape, which becomes the areal footprint of the conductive material layer 101, 201. The electrically conductive fibers may be oriented such that the longitudinal axes of each of the fibers is substantially (e.g., within 20 degrees, or within 10 degrees parallel to the x-y plane of the adhesive layer 112, 212; or 114, 214. In some embodiments, the electrically conductive fibers may provide enhanced electrical conductivity in the x-y plane of the adhesive layer. The adhesive matrix material may comprise any suitable polymer, for example, the adhesive matrix material may comprise an acrylic polymer matrix material or a silicone polymer matrix material. The conductive adhesive layer 112, 212; or 114, 214 may comprise a medical grade adhesive that requires no hydrogel or Ag/AgCl to get a signal, sold under the trademark FLEXcon® OMNI-WAVE™ (available from FLEXcon located in Spencer, Massachusetts, USA); or, alternatively, ARcare® 8006 electrically conductive adhesive composition manufactured and sold by Adhesives Research, Inc. (Glen Rock, PA, USA).

In some embodiments, the electrically conductive adhesive layer 112, 212; or 114, 214 may not include a plurality of electrically conductive particles that provide enhanced electrical/heat conductivity in the x-y plane of the adhesive layer. In other embodiments, the anisotropic material layer may not be present in the transducer 100, 100A, 200, 200A, such that the one or more electrically conductive adhesive layers 112, 212; or 114, 214 are the only conductive material layer(s) 101, 201.

The one or more conductive material layer(s) 101, 201, which may include the anisotropic material layer 106, 206, the electrically conductive skin contact adhesive layer 112, 212, or the electrically conductive upper adhesive layer 114, 214, or a combination thereof, may take any desired shape. For example, as shown in FIGS. 1A, and 1E, a perimeter ring 110 of the conductive material layer 101, which represents the outer perimeter 110A of the anisotropic material layer 106 and the outer perimeter 110B of the electrically conductive skin contact adhesive layer 112, may have a substantially square or rectangular shape, or substantially square or rectangular shape with rounded corners. As another example, as shown in FIGS. 2A and 2C, an outer perimeter 210 of the conductive material layer 201, which represents the outer perimeter 210A of the anisotropic material layer 206 and the outer perimeter 210B of the electrically conductive skin contact adhesive layer 22 may have a circular, oval, ovoid, ovaloid, or elliptical shape. In FIGS. 1A, 1E, 2A, and 2C, the outer perimeter 110, 210 of the anisotropic material layer 106, 206 and the electrically conductive skin contact adhesive layer 112, 212 may define an areal footprint of the conductive material layer(s) 101, 201. Although the outer perimeter 110, 210 in FIGS. 1A, 1E, 2A, and 2C may represent the outer perimeters 110A/110B, 210A/210B of both the anisotropic material layer 106, 206 and the electrically conductive skin contact adhesive layer 112, 212, in other embodiments the outer perimeter 110, 210 may correspond to only one of the anisotropic material layer 106, 206 or the electrically conductive adhesive layer(s) 112, 212; 114, 214. This may be the case where the outer perimeter 110A, 210A of the anisotropic material layer 106, 206 is different from the outer perimeter 110B, 210B of the electrically conductive adhesive layer(s) 112, 212; 114, 214.

Turning to FIGS. 1A-2E, the transducer 100, 100A, 200, 200A may further include a non-conductive material border 108, 108A, 208, 208A at least partially disposed on a front facing side of the conductive material layer(s) 101, 201 and over at least a portion of the outer perimeter 110, 210 of the conductive material layer(s) 101, 201. That is, the transducer 100, 100A, 200, 200A may include the non-conductive material border 108, 108A, 208, 208A at least partially disposed on the front facing side of the anisotropic material layer 106, 206 and over at least a portion of the outer perimeter 110A, 210A of the anisotropic material layer 106, 206 and/or over the outer perimeter 110B, 210B of the electrically conductive adhesive layer(s) 112, 212; 114, 214. The non-conductive material border 108, 108A, 208, 208A may be electrically non-conductive.

As illustrated in FIGS. 1A, 1E, 2A, and 2C, the non-conductive material border 108, 108A, 208, 208B may be generally ring-shaped or annular shaped, having an inner edge 116, 216 and an outer edge 118, 218. When viewed in a direction perpendicular to the front face 106A, 206A of the anisotropic material layer 106, 206, the inner edge 116, 216 may overlap a portion of the front face 106A, 206A of the anisotropic material layer 106, 206, and the outer edge 118, 218 may extend outside the outer perimeter 110A, 210A of the anisotropic material layer 106, 206. In an example, the inner edge 116, 216 of the non-conductive material border 108, 108A, 208, 208A may overlap the front face 106A, 206A of the anisotropic material layer 106, 206 along an entire length of the inner edge 116, 216, and the outer edge 118, 218 of the non-conductive material border 108, 108A, 208, 208A may extend outside the outer perimeter 110A, 210A of the anisotropic material layer 106, 206 along an entire length of the non-conductive material border 108, 208, such that all of the outer perimeter 110A, 210A of the anisotropic material layer 106, 206 is covered by the non-conductive material border 108, 108A, 208, 208A. For example, the outer edge 118, 218 of the non-conductive material border 108, 108A, 208, 208A may extend at least 1.0 mm, or at least 1.2 mm, or at least 1.5 mm, or at least 2.0 mm, or more, outside of the outer perimeter 110A, 210A of the anisotropic material layer 106, 206.

As illustrated in FIGS. 1A-2E, the inner edge 116, 216 and outer edge 118, 218 of the non-conductive material border 108, 108A, 208, 208A may have a similar overlapping arrangement with respect to the front face (e.g., 112A, 212A) and to the outer perimeter (e.g., 110B, 210B) of the electrically conductive adhesive layer(s) 112, 212; 114, 214 as described at length above with respect to the front face 106A, 206A and outer perimeter 110A, 210A of the anisotropic material layer 106, 206. When viewed in cross-section, as in FIGS. 1B-1D, 1F, 1H, 2B, and 2D, the non-conductive material border 108, 108A, 208, 208A may have a different shape. For example, in FIGS. 1B and 2B, the non-conductive material border 108, 208 may have an “L” shape. For example, in FIGS. 1C and 1D, the non-conductive material border 108 may have a “C” shape. For example, in FIGS. 1F and 2D, the non-conductive material border 108A, 208A may have an S″ shape or a “Z” shape. Additionally, as shown in FIG. 1H, a portion of flexible layer 108 between the anisotropic material layer 106 and the patient may have an angled profile on the inner edge 116H relative to the anisotropic material layer 106. For each of these constructs, a portion of the non-conductive material border 108, 108A, 208, 208A functions as an edge seal having an outwardly facing surface of the edge seal portion 118, 118A, 218, 218A of the non-conductive material border 108, 108A, 208, 208A.

As illustrated in FIG. 1C, the “inside” of the “C” shape of the non-conductive material border 108, 208 may include both the outer perimeter 110A, 210A of the anisotropic material layer 106, 206 and the outer perimeter 110B, 210B of the electrically conductive adhesive layer 112, 212. As illustrated in FIG. 1D, the “inside” of the “C” shape of the non-conductive material border 108, 208 may include only the outer perimeter 110B, 210B of the electrically conductive adhesive layer 112, 212. Although not illustrated, the “inside” of the “C” shape of the non-conductive material border 108, 208 may include only the outer perimeter 110A, 210A of the anisotropic material layer 106, 206.

As illustrated in FIGS. 1A-2E, the inner edge 116, 216 of the non-conductive material border 108, 108A, 208, 208A may extend a distance 120, 220 of at least 0.2 mm, at least 0.5 mm, at least 1 mm, at least 2 mm, at least 3 mm, or more, inward from the outer perimeter 110A, 210A of the anisotropic material layer 106, 206. In an embodiment, the distance 120, 220 may be less than at most 0.6 mm, at most 1 mm, at most 2 mm, at most 3 mm, at most 4 mm, or more. In an embodiment, the distance 120, 220 may be greater than at least 0.2 mm and less than at most 3 mm. In an embodiment, the distance 120, 220 may be 1 mm, or approximately 1 mm. In an embodiment, the distance 120, 220 may be 2 mm, or approximately 2 mm.

In some embodiments, the outer edge 118, 218 of the non-conductive material border 108, 208 may extend a distance 122, 222 outside of the outer perimeter 110A, 210A of the anisotropic material layer 106, 206. In some such embodiments (for example, as described herein for the “L” shape or “C” shape constructs), the outer edge 118, 218 of the non-conductive material border 108, 208 may extend a distance 122, 222 of greater than at least 0.2 mm, at least 0.4 mm, at least 0.5 mm, at least 1 mm, at least 2 mm, at least 3 mm, at least 5 mm, or more, outside of the outer perimeter 110A, 210A of the anisotropic material layer 106, 206. In some embodiments, the distance 122, 222 may be less than at most 0.6 mm, at most 1 mm, at most 2 mm, at most 3 mm, at most 5 mm, or more. In an embodiment (for example, the “L” shape or “C” shape configurations described herein), the distance 122, 222 may be greater than at least 0.2 mm and less than at most 1 mm. For example, the distance 122, 222 may be 0.5 mm, or approximately 0.5 mm. In some embodiments (for example, as described for the “Z” shape construct), the outer edge 118, 218 of the non-conductive material border 108, 208 may extend a distance equal to the sum of the distances 122, 222 and 123, 223 outside of the outer perimeter 110A, 210A of the anisotropic material layer 106, 206. In some such embodiments, the outer edge 118, 218 of the non-conductive material border 108, 208 may extend a distance equal to the sum of the distances 122, 222 and 123, 223 that is greater than at least 0.2 mm, at least 0.4 mm, at least 0.5 mm, at least 1 mm, at least 2 mm, at least 3 mm, at least 5 mm, at least 10 mm, or more, outside of the outer perimeter 110A, 210A of the anisotropic material layer 106, 206. For example (and with regard to the “Z” shape configuration described herein, FIGS. 1E-1G and 2C-2E, the combined distance 122, 222 and 123, 223 may be greater than at least 0.4 mm and less than at most 10 mm. For example, the distance 122, 222 may be 5 mm, or approximately 5 mm.

Referring to FIGS. 1E-1G and 2C-2E, the non-conductive material border 108A, 208A may include a base edge 117, 217 extending outside the outer perimeter 110A, 210A of the anisotropic material layer 106, 206. In these examples (“Z” shaped construct), the base edge 117, 217 is also the outer edge 118, 218 of the non-conductive material border 108A, 208A. For the “L” and “C” shape constructs of FIGS. 1B-1D and 2B, the outwardly facing surface of the edge seal portion 118, 118A, 218, 218A of the non-conductive material border 108A, 208A is the outer edge 118, 218 of the non-conductive material border 108A, 208A. For the “Z” shape constructs of FIGS. 1E-1G and 2C-2E the outwardly facing surface of the edge seal portion of the non-conductive material border 108A, 208A is labelled 118A, 218A. The base edge 117, 217 (which is also the outer edge 118, 218) of the non-conductive material border 108A, 208A may extend further than the outwardly facing surface of the edge seal portion 118A, 218A of the non-conductive material border 108A, 208A outside the outer perimeter 110A, 210A of the anisotropic material layer 106, 206. In an example, the inner edge 116, 216 of the non-conductive material border 108A, 208A overlaps the front face 106A, 206A of the anisotropic material layer 106, 206 along an entire length of the inner edge 116, 216, and the outwardly facing surface of the edge seal portion 118A, 218A and the base edge 117, 217 (which is also the outer edge 118, 218) of the non-conductive material border 108A, 208A extend outside the outer perimeter 110A, 210A of the anisotropic material layer 106, 206 along an entire length of the non-conductive material border 108A, 208A, such that all of the outer perimeter 110A, 210A of the anisotropic material layer 106, 206 and a portion of the surface 124A, 224A of the substrate 102, 202 are covered by the non-conductive material border 108A, 208A. The base edge 117, 217 of the non-conductive material border 108A, 208A, which, in these examples, is also the outer edge 118, 218 of the non-conductive material border 108A, 208A, may extend a distance 123, 223 of at least 1 mm, at least 2 mm, at least 3 mm, at least 5 mm, at least 10 mm, or more, outside of the outwardly facing surface of the edge seal portion 118A, 218A and along the surface 124A, 224A of the substrate 102, 202, such that the base edge 117, 217 or outer edge 118, 218 overlaps a portion of the surface 124A, 224A of the substrate 102, 202.

Referring to FIGS. 1E-1G and 2C-2E, in an example, a summation of the distance 122, 222 and the distance 123, 223 (i.e., a distance from the base edge 117, 217 or outer edge 118, 218 to the outside edge of the outer perimeter 110A, 210A of the anisotropic material layer 106, 206) may be at least 1 mm, at least 1.2 mm, at least 1.5 mm, at least 2 mm, at least 3 mm, at least 5 mm, at least 10 mm, or more. In an example, the summation of the distance 122, 222 and the distance 123, 223 may be less than at most 1.5 mm, at most 1.6 mm, at most 2 mm, at most 3 mm, at most 5 mm, at least 10 mm, or more. In an example, the summation of the distance 122, 222 and the distance 123, 223 may be more than at least 1 mm and less than at most 5 mm, such as, for example, more than at least 1.2 mm and less than at most 3.0 mm. In an example, the summation of the distance 122, 222 and the distance 123, 223 may be 2 mm, or approximately 2 mm.

FIGS. 1G and 2E depict zoom-in views of adhesive areas 1G and 2E of the example transducer apparatuses 100A and 200A of FIGS. 1F and 2D, respectively. As illustrated in FIGS. 1G and 2E, a non-conductive adhesive layer 133, 233 may be positioned to adhere the non-conductive material border 108A, 208A to the transducer 100A, 200A. For the example, the adhesive layer 133, 233 may be positioned at one or more of: between the non-conductive material border 108A, 208A and an edge of the stack of the conductive material layer 101, 201, the anisotropic material layer 106, 206, and the adhesive layer 112, 212; between the non-conductive material border 108A, 208A and the front face (e.g., 112A, 212A of the electrically conductive adhesive layer 112, 212; and/or between the non-conductive material border 108A, 208A and the surface 124A, 224A of the substrate 102, 202. Although not illustrated, an adhesive layer may be positioned similarly for the non-conductive material border 108, 208 of FIGS. 1B-1D, 1H, and 2B.

As illustrated in FIGS. 1G and 2E, the non-conductive material border 108, 108A, 208, 208A may have a thickness 125, 225 more than at least 0.1 mm and less than at most 3 mm, such as, for example, more than at least 0.2 mm and less than at most 1 mm. The thickness 125, 225 may be 0.5 mm or approximately 0.5 mm. In some embodiments, the thickness 125, 225 may be the same as the thickness 122, 222.

In some embodiments, the non-conductive material border 108, 108A, 208, 208A may be contoured in a gradient or stepwise manner to cover a full thickness of the edge of a stack of layers comprising the anisotropic material layer (such as, for example, a stack comprising the conductive material layer 101, 201, the anisotropic material layer 106, 206, and the adhesive layer 112, 212) by placement of at least one additional material adjacent to the edge of the stack and adjacent to the front face of the substrate, the additional material protruding in a forward direction by an amount less than the full thickness of the edge of the stack. For example, a surrounding ring of foam having a smaller thickness than the full thickness of the edge of the stack may provide a gradient or stepwise transition for the edge seal portion of the non-conductive material border connecting between the top of the stack (e.g., at the front face of the stack) and the front face of the substrate adjacent to the base of the stack. The additional material (e.g., foam) may or may not be non-conductive.

In an example, the non-conductive material border 108, 108A, 208, 208A may be, or may comprise, a non-conductive adhesive. The non-conductive adhesive may be a medical adhesive. The non-conductive adhesive may be sprayed onto or otherwise applied to the rest of the transducer 100, 100A, 200, 200A to form the non-conductive material border 108, 108A, 208, 208A. As described above, the non-conductive adhesive may be applied such that all of the outer perimeter 110, 210 of the conductive material layer 101, 201 (e.g., all of the outer perimeter 110A, 110B of the anisotropic material layer 106, 206 and/or all of the outer perimeter 110B, 210B of the electrically conductive adhesive layer(s) 112, 212; 114, 214) is covered by the non-conductive adhesive. In another embodiment, the non-conductive adhesive may be applied only outside of the outer perimeter 110, 210 of the conductive material layer 101, 201, for example, starting at the outer perimeter 110, 210 and extending outside of the outer perimeter 110, 210 to form an adhesive “skirt”; or starting outside the outer perimeter 110, 210 and extending further outside of the outer perimeter 110, 210 to form an adhesive “skirt”. The latter approach may be advantageous compared to relying on the area of bandage outside of the outer perimeter 110, 210, particularly if the adhesive used for the “skirt” is less irritating on the skin than the bandage adhesive. The same adhesive “skirt” may be achieved in practice by coating a layer (or area with a central void) of non-conductive adhesive over a portion of the front face 103, 203 of the substrate bandage 102, 202 prior to applying the electrode assembly comprising the conductive material layer 101, 201 onto the substrate 102, 202. In this method of construction, the layer (or area with a central void) of non-conductive adhesive extends out from beneath the anisotropic material layer 106, 206, extending beyond the outer perimeter 110, 210 thereby forming the adhesive “skirt”.

In another example, the non-conductive material border 108, 108A, 208, 208A may comprise a tape, bandage, plaster, or foam. In particular, the non-conductive material border 108, 108A, 208, 208A may comprise an electrical tape or a non-conductive medical tape. In some embodiments, the non-conductive material border 108, 108A, 208, 208A may comprise foam. In some embodiments, the non-conductive material border 108, 108A, 208, 208A may be an adhesive coated foam. In some embodiments, the adhesive layer is coated on the front face (skin-facing side) of the foam layer. In some embodiments, the adhesive layer is a biocompatible adhesive. In some embodiments, the non-conductive material border 108, 108A, 208, 208A may be 3M™ Tegaderm™ Transparent Film Dressing Frame, which may include adhesive on one side facing the patient's body and facing away from the substrate 102, 202). In particular, with the foam material, the non-conductive material border 108, 108A, 208, 208A may be used as a barrier for hydrogel to prevent substrate absorbing moisture from the hydrogel.

The non-conductive tape or bandage or adhesive coated foam may be applied as an “o-ring” to seal the outer edge of the anisotropic material layer 106, 206 and/or the electrically conductive adhesive layer(s) 112, 212; 114, 214. In an embodiment, for example, as shown in FIGS. 1B, 1H, and 2B, the non-conductive tape or bandage or adhesive coated foam may adhere to the front face 106A, 206A, or on the front facing side, of the anisotropic material layer 106, 206 within the outer perimeter 110A, 210A of the anisotropic material layer 106, 206 and also adhere to the substrate 102, 202 outside of the outer perimeter 110A, 210A of the anisotropic material layer 106, 206. In another embodiment, for example, as shown in FIG. 1C, the non-conductive tape or bandage or adhesive coated foam may adhere to the front face 106A, or on the front facing side, of the anisotropic material layer 106 within the outer perimeter 110A of the anisotropic material layer 106 and also be folded to adhere to the back face 106B, or on the back facing side, of the anisotropic material layer 106. In another embodiment, for example, as shown in FIG. 1D, the non-conductive tape or bandage or adhesive coated foam may adhere to the front face 112A of the electrically conductive skin contact adhesive layer 112 within the outer perimeter 110B of the electrically conductive skin contact adhesive layer 112 and also be folded to adhere to the back face 112B of the electrically conductive skin contact adhesive layer 112. In another embodiment, for example, as shown in FIGS. 1F and 2D, the non-conductive tape or bandage or adhesive coated foam may adhere to the front face 106A, 206A, or on the front facing side, of the anisotropic material layer 106, 206 within the outer perimeter 110A, 210A of the anisotropic material layer 106, 206 and also adhere to the substrate 102, 202 outside of the outer perimeter 110A, 210A of the anisotropic material layer 106, 206. In some embodiments, a one-sided or two-sided non-conductive tape, band-aid, plaster, or adhesive coated foam may be added around the perimeter ring 110, 210.

The non-conductive material border 108, 108A, 208, 208A may prevent or protect against a short circuit occurring between the transducer 100, 100A, 200, 200A and an adjacent transducer positioned on a subject's body, even if one or both of the transducers have been cut. The non-conductive material border 108, 108A, 208, 208A is a border defined by a physical barrier (i.e., the non-conductive material). The non-conductive material border 108, 108A, 208, 208A may surround an areal exclusion zone of the transducer 100, 100A, 200, 200A containing at least the areal footprint of the anisotropic material layer 106, 206. The non-conductive material border 108, 108A, 208, 208A may seal the outer edge of the anisotropic material layer 106, 206 from electrical contact with other transducers in its vicinity.

In FIGS. 1A-2E, the transducer 100, 100A, 200, 200A may further include one or more electrically conductive adhesive layers. For example, the transducer 100, 100A, 200, 200A may include an electrically conductive adhesive layer 112, 212 located on the front face 106A, 206A of the anisotropic material layer between the anisotropic material layer 106, 206 and the front face 124B, 224B of the non-conductive material border 108, 208. Additionally, or alternatively, the transducer 100, 100A, 200, 200A may include the electrically conductive upper adhesive layer 114, 214 located between the array of at least one electrode 104, 204 and the back face 106B, 206B of the anisotropic material layer 106, 206. The upper adhesive layer 114, 214 may extend from the substrate 102, 202 to the anisotropic material layer 106, 206. Alternatively, the upper adhesive layer 114, 214 may simply coat the front face of the array of at least one electrode 104, 204 facing the anisotropic material layer 106, 206.

In an example, as shown in FIGS. 1B, 1C, 1F, 1H, 2B, and 2D, the non-conductive material border 108, 108A, 208, 208A covers a full thickness 126, 226 of the anisotropic material layer 106, 206 in the direction perpendicular to the front face 106A, 206A of the anisotropic material layer 106, 206. As shown in FIGS. 1B-1D, 1F, 1H, 2B, and 2D, the non-conductive material border 108, 108A, 208, 208A may cover a full thickness 150, 250 of the electrically conductive skin contact adhesive layer 112, 212 in the direction perpendicular to the front face 112A, 212A of the electrically conductive skin contact adhesive layer 112, 212. As shown in FIGS. 1B, 1F, 1H, 2B and 2D, the non-conductive material border 108, 108A, 208, 208A may cover a full thickness of the electrically conductive upper adhesive layer 114, 214 in the direction perpendicular to the front face 103 of the substrate 102. In addition, as shown in FIGS. 1B, 1F, 1H, 2B, and 2D, the non-conductive material border 108, 108A, 208, 208A may be adhered to the front face 103, 203 of the substrate 102, 202. As such, the non-conductive material border 108, 108A, 208, 208A may extend from the front face 103, 203 of the substrate 102, 202 to the very front of the transducer 100, 100A, 200, 200A, thereby covering the full thickness of all conductive material layers 101, 201. As constructed, the transducer 100, 100A, 200, 200A may present exposed surfaces facing in the forward-facing direction. In the transducer 100, 100A, 200, 200A, the forward-facing surfaces of the substrate 102, 202, non-conductive material border 108, 108A, 208, 208A, and conductive adhesive layer 112, 212 are surfaces 124A/224A, 124B/224B and 124C/224C, respectively. The dimensions of various components of the transducer 100, 100A, 200, 200A in FIGS. 1A-2E are not shown to scale, and the transducer 100, 100A, 200, 200A may be substantially flat such that surfaces 124A-124C, 224A-224C of multiple components of the transducer 100, 100A, 200, 200A contact the subject's body upon placement of the transducer 100, 100A, 200, 200A on the subject's body.

### Example Second Transducers

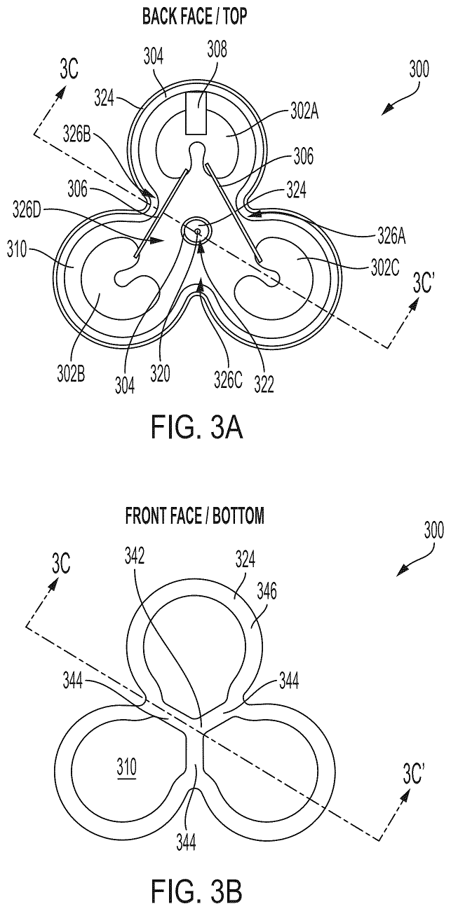

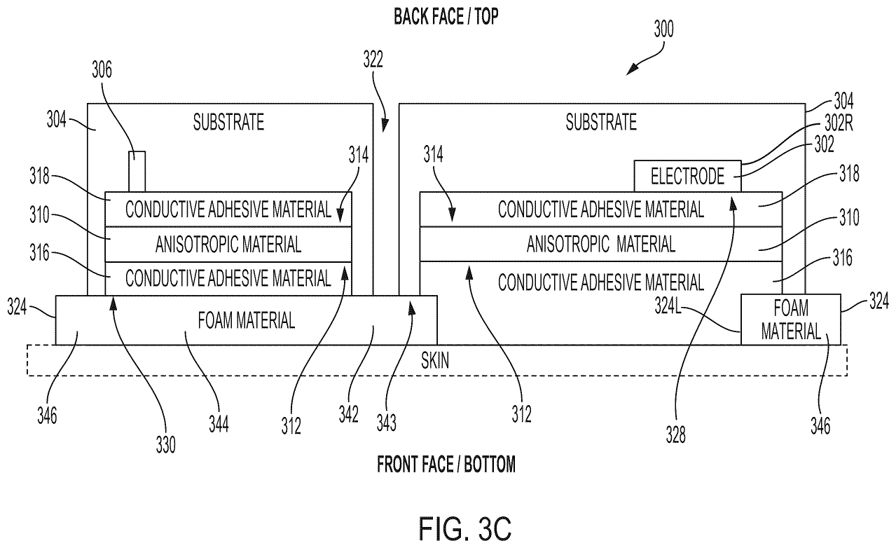

FIGS. 3A and 3B depict a top plan view and a bottom plan view, respectively, of an example transducer apparatus 300 according to some embodiments. FIG. 3C depicts a cross-section view of the example transducer apparatus 300 of FIGS. 3A and 3B taken across the section 3C-3C′ according to some embodiments. In FIG. 3A, the substrate 304, if present, would cover the back side of the transducer array, obscuring the view of the electrode elements 302. Accordingly, the substrate 304 is only shown peripherally in FIG. 3A so as to illustrate the components beneath the substrate 304.

As shown in FIGS. 3A and 3B, the transducer apparatus 300 may have a plurality of table-tennis bat/paddle shapes extending from a center, or have a shape comprised of a plurality of substantially circular shapes connected about a center. The transducer apparatus 300, as illustrated, may include a plurality of electrode elements 302 (e.g., 302A, 302B, 302C), a substrate 304, and an anisotropic material layer 310. The transducer apparatus 300 may include one or more blank spaces 326 (e.g., 326A-326D), which do not overlap with any of the electrode elements 302. At least part of one or more of the blank spaces 326 may be a relief region, defined herein as either: (1) void regions of the transducer apparatus 300 that are fully uncovered or fully uncovered other than the transducer substrate and/or an anisotropic material layer (with or without conductive adhesive layer(s) (e.g., 316) and/or a conductive layer (e.g., 318), FIG. 3C); (2) non-adhesive regions comprising a medication substrate capable of receiving, absorbing, and/or holding a topical medication applied thereto; or (3) medication regions of the transducer apparatus comprising a medication substrate and a topical medication integrated therein or thereon, which may be used to administer a topical medication to an area of the subject's skin. These relief regions may, optionally, have no exposed adhesive present.

The plurality of electrodes elements 302 may be spaced about a centroid 320 of the transducer apparatus 300, and the blank spaces 326 may each be located between two adjacent electrodes and around the centroid 320. In some embodiments, the transducer apparatus 300 may have an alternating pattern of electrodes 302 and blank spaces 326. In other embodiments, non-alternating rotational patterns of electrodes 302 and blank spaces 326 may be used. The electrodes 302 may be electrically coupled together via one or more printed circuit board (PCB) connector(s) 306 or wire(s). The transducer apparatus may be electrically coupled to a voltage generator via a lead connector 308. The PCB connector(s) 306 and lead connector 308 are not electrodes and are non-adhesive regions. Although three electrodes 302 and four blank spaces 326 are shown in FIGS. 3A and 3B, other embodiments may include different numbers of electrodes 302, blank spaces 326, or both in the array. For example, some embodiments may include six electrodes 302 and seven blank spaces 326; or five electrodes 302 and six blank spaces 326; or four electrodes 302 and five blank spaces 326; or three electrodes 302 and four blank spaces 326; or two electrodes 302 and three blank spaces 326.

As shown in FIG. 3A, each electrode 302 of the array may be u-shaped, substantially u-shaped, jelly-bean or kidney-bean shaped, substantially jelly-bean or kidney-bean shaped, horseshoe shaped, or substantially horseshoe shaped. In some embodiments, the end-points of the u-shape may be close together. In other embodiments, the end-points of the u-shape may have a gap in between. In some embodiments, the open end of the u-shaped electrode 302 may face the centroid 320 of the transducer apparatus 300. In addition, a centroid of each electrode 302 may be spaced substantially equidistant from the centroid 320 of the transducer apparatus 300. Each electrode 302 may have a substantially similar shape The electrodes 302 may be spaced substantially equidistant from each other about the centroid 320 of the array. The electrodes 302 may be spaced substantially equidistant from the centroid 320 of the array and/or may be spaced substantially equidistant from each other.

The transducer apparatus 300 may include an anisotropic material layer 310 directly or indirectly electrically coupled to the plurality of electrodes 302 and located on a front face 328 (FIG. 3C) of the electrodes 302 which may be configured to face the subject's body. The anisotropic material layer 310 may include any of the features described in further detail above with reference to the anisotropic material layer 106, 206. The anisotropic material layer 310 may take any of the forms and may include any of the features described in further detail below with reference to the anisotropic material layer 310 of FIG. 3C.

The anisotropic material layer 310 may be disposed over the plurality of electrodes such that the anisotropic material layer 310 covers the electrode elements 302A-302C and, optionally, the at least one blank space 326 (e.g., void space) in the array. In some embodiments, the anisotropic material layer 310 may be disposed over the plurality of electrodes to cover the electrode elements 302A-302C and blank space 326A-326D in the array. In some embodiments, the anisotropic material layer 310 may not extend outward all the way to the edge of the substrate layer 304. The anisotropic material layer 310 may include a hole 322 in, or substantially in, the center or the middle of the anisotropic material layer 310. The hole 322 may pass through the front face 312 and the back face 314 of the anisotropic material layer 310, thereby facilitating flexibility and pliability of the anisotropic material layer 310. The hole 322 may be circular, oval, or ovoid, or substantially circular oval, or ovoid in shape. The hole 322 may be positioned at or near the centroid 320 of the transducer apparatus 300. For example, the centroid 320 of the transducer apparatus 300 may be positioned at the center of the hole 322. Furthermore, the centroid of each electrode 302 may be spaced substantially equidistant from the hole 322. The PCB connector(s) 306 or wire(s) may not, in some embodiments, pass over the hole 322 of the anisotropic material layer 310 and/or may be positioned equidistantly from the hole 322 of the anisotropic material layer 310.

The transducer apparatus 300 may further include a foam material layer 324 directly or indirectly coupled to the anisotropic material layer 310 and located on a front face 312 of the anisotropic material layer 310. The foam material layer 324 may be configured to contact the subject's body. The foam material layer 324 may cover at least the hole 322 formed in the anisotropic material layer 310. In some embodiments, the foam material layer 324 may include a hole covering portion 342, positioned over the hole 322 in the anisotropic material layer 310 (FIG. 3C). The hole covering portion 342 may be dimensioned larger than the hole 322 in the anisotropic material layer 310. The foam material layer may include a perimeter portion 346 covering a perimeter of the anisotropic material layer 310. In some embodiments, the anisotropic material layer 310 may not extend outward all the way to the edge of the foam material layer 324. In some embodiments, the perimeter portion 346 of the foam material layer 324 may cover up to the edge of the perimeter of the anisotropic material layer 310. The perimeter of the perimeter portion of the foam material layer 324 may be larger than the perimeter of the anisotropic material layer 310.

As further shown in FIG. 3B, the foam material layer 324 may cover at least the hole 322 on the side of the transducer apparatus 300 facing the subject's body. The perimeter of the perimeter portion 346 of the foam material layer 324 may be larger than the perimeter of the anisotropic material layer 310. The perimeter of the foam material layer 324 may have substantially the same shape as the perimeter of the anisotropic material layer 310. The foam material layer 324 may include at least one connection portion 344 connecting the hole covering portion 342 and the perimeter portion 346. The at least one connection portion 344 may cover the front side 312 of the anisotropic material layer 310. When viewed from a direction perpendicular to the front face 312 of the anisotropic material layer 310, the at least one connection portion 344 may be located coincident with the blank spaces 326, except on the skin-facing side of the anisotropic material layer 310. The foam material layer 324 may cover at most 30%, or at most 40%, or at most 50%, of the anisotropic material layer 310.

The substrate 304 may be configured for attaching a front side of the transducer 300 to a subject's body. Suitable materials for the substrate 304 include, for example, cloth, foam, flexible plastic, and/or a conductive medical gel. The transducer 300 may be affixed to the subject's body via the substrate 304 (e.g., via an adhesive layer and/or a conductive medical gel). The substrate 304 may be an adhesive bandage. The adhesive layer that contacts the subject's skin may be present around the outer perimeter of the array of electrodes 302, and/or may be present in a central-middle area defined by the electrodes 302 (or between one or more gaps between electrodes).

The transducer apparatus 300 may include the hole 322 that extends through each layer of the transducer apparatus 300 except for the foam layer 324. The transducer apparatus 300 may include an anisotropic material layer 310. As shown in FIG. 3C, the anisotropic material layer 310 may have a front face 312 and a back face 314, wherein the back face 314 faces the array of electrode elements 302. The anisotropic material layer 310 has anisotropic thermal properties and/or anisotropic electrical properties. If the anisotropic material layer 310 has anisotropic thermal properties (for example, greater thermal conductivity in the plane of the layer 310 than through the plane of the layer 310), then the layer 310 spreads the heat out more evenly over a larger surface area. If the anisotropic material layer 310 has anisotropic electrical properties (for example, greater electrical conductivity in the plane of the layer 310 than through the plane of the layer 310), then the layer 310 spreads the current out more evenly over a larger surface area. In each case, this lowers the temperature of the hot spots and raises the temperature of the cooler regions when a given AC voltage is applied to the array of electrode elements 302. Accordingly, the current may be increased (thereby increasing the therapeutic effect) without exceeding the safety temperature threshold at any point on the subject's skin.

In some embodiments, the anisotropic material layer 310 may be anisotropic with respect to electrical conductivity properties. In some embodiments, the anisotropic material layer 310 may be anisotropic with respect to thermal conductivity properties. In some embodiments, the anisotropic material layer 310 may be anisotropic with respect to both electrical conductivity properties and thermal conductivity properties.

The anisotropic thermal properties may include directional thermal properties. Specifically, the anisotropic material layer 310 may have a first thermal conductivity in a direction that is perpendicular to its front face (skin-facing surface) 312 that is different from a thermal conductivity of the anisotropic material layer 310 in directions that are parallel to the front face 312. For example, the thermal conductivity of the anisotropic material layer 310 in directions parallel to the front face 312 may be more than two times higher than the first thermal conductivity. In some preferred embodiments, the thermal conductivity in the parallel directions may be more than ten times higher than the first thermal conductivity. For example, the thermal conductivity of the sheet in directions that are parallel to the front face 312 may be more than: 3.5 times, 2 times, 3 times, 5 times, 30 times, 20 times, 300 times, 200 times, or even more than 3,000 times higher than the first thermal conductivity.

The anisotropic electrical properties may also include directional electrical properties. Specifically, the anisotropic material layer 310 may have a first electrical conductivity (or, conversely, resistance) in a direction that is perpendicular to its front face 312 that is different from an electrical conductivity (or resistance) of the anisotropic material layer 310 in directions that are parallel to the front face 312. For example, the resistance of the anisotropic material layer 310 in directions parallel to the front face 312 may be less than the first resistance. In some embodiments, the resistance in the parallel directions is less than half of the first resistance or less than 30% of the first resistance. For example, the resistance of the anisotropic material layer 310 in directions that are parallel to the front face 312 may be less than: 75%, 50%, 40%, 30%, 20%, 30%, 5%, 3%, 0.5%, or even less than 0.3% of the first resistance.

In some embodiments (e.g., when the anisotropic material layer 310 is a sheet of pyrolytic graphite), the anisotropic material layer 310 may have both anisotropic electrical properties and anisotropic thermal properties.

The anisotropic material layer 310 may comprise graphite (e.g., a sheet of graphite or a graphite sheet). Examples of suitable forms of graphite may include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2030A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). In alternative embodiments, conductive anisotropic materials other than graphite may be used instead of graphite.

In some embodiments, the anisotropic material layer 310 may be a sheet of pyrolytic graphite. Thermal conductivity of pyrolytic graphite sheets in directions that are parallel to the front face 312 of those sheets may typically be more than 50 times higher than the thermal conductivity of those sheets in directions that are perpendicular to the front face 312. Electrical resistivity of pyrolytic graphite sheets in directions that are parallel to the front face 312 of those sheets may typically be less than 2% of the electrical resistivity of those sheets in directions that are perpendicular to the front face 312.

While an anisotropic material layer comprised of graphite may reduce the concentration of heat and/or current at locations directly under the individual electrode elements-thus reducing the amount or severity of irritation, if any, that occurs on the subject's skin-such anisotropic material may be relatively inflexible. In other words, when pressed against a non-flat surface, the anisotropic material layer 310 may be at risk of cracking and/or breaking. As such, the anisotropic material layer 310 may not easily conform to a subject's body, which may typically be non-planar over a large area or which may bend and change shape as the subject moves. Additionally, due to a non-planar surface on a subject and/or a surface of the subject bending and changing shape, the anisotropic material layer 310 may deform or even crack, resulting in the transducer array 300 producing a less than desired electric field. Obtaining a flush contact surface between the electrode element(s) 302 and the subject's skin may improve the effectiveness of the treatment. Accordingly, the anisotropic material layer 310 may include the hole 322 substantially in the center of the anisotropic material layer 310, passing through the front face 312 and the back face 314. In this way, the hole 322 may facilitate flexibility and pliability of the anisotropic material layer 310. The hole 322 in the anisotropic material layer 310 may further facilitate generation of an effective electric field and obtaining a flush contact between the transducer 300 and the subject's skin.

Referring to FIG. 3C, the transducer 300 may further include at least one layer of conductive adhesive material 316 disposed on a front facing side of the anisotropic material layer 310. In some embodiments, the at least one layer of conductive adhesive material 316 may be disposed on the front face 312 of the anisotropic material layer 310. The conductive adhesive material 316 may include the hole 322 which may be aligned with the hole 322 through the anisotropic material layer 310. The at least one layer of conductive adhesive material 316 may have a biocompatible front surface. In some embodiments, there may only be a single layer of conductive adhesive material 316, and that single layer (the front layer) may be biocompatible. In some embodiments, there may be more than one layer of conductive adhesive material 316, in which case only the front layer may be biocompatible, or the front layer and one or more other layers may be biocompatible. In some embodiments, the front layer of conductive adhesive material 316 may be configured to ensure good electrical contact between the device and the body. In some embodiments, the front layer of conductive adhesive material 316 may be configured to ensure good adhesion between the anisotropic material layer 310 and the foam material layer 324. In some embodiments, the front layer of conductive adhesive material 316 may cover the entire front face 312 of the anisotropic material layer 310. The front layer of conductive adhesive material 316 may be the same size (area) or larger than the anisotropic material layer 310. In some embodiments, the front layer of conductive adhesive material 316 may comprise hydrogel. In these embodiments, the hydrogel may have a thickness between 50 and 2,000 μm. In some embodiments, the front layer of conductive adhesive material 316 may comprise a conductive adhesive composite as further disclosed herein.

Referring to FIG. 3C, the transducer 300 may further include at least one conductive material layer 318. A conductive material layer 318 may be positioned between the array of electrode elements 302 and the back face 314 of the anisotropic material layer 310 facing the array. The conductive material layer 318 may facilitate the electrical contact between the array of electrode elements 302 and the back face 314 of the anisotropic material layer 310. The conductive material layer 318 may include the hole 322 and may be aligned with the hole 322 through the anisotropic material layer 310 and the conductive adhesive material 316. In some embodiments, the conductive material layer 318 may be a layer of hydrogel. In other embodiments, a different conductive material (e.g., conductive grease, conductive adhesives, conductive tape, etc.) may be used. For example, the conductive material layer 318 may comprise a conductive adhesive composite as further disclosed herein.

In some embodiments, the at least one layer of conductive adhesive material 316 and/or the layer of conductive material 318 may be a single layer of non-hydrogel conductive adhesive such as the developmental product FLX068983-FLEXcon® OMNI-WAVE™ TT 200 BLACK H-502 350 POLY H-9 44PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Non-hydrogel conductive adhesives may comprise a waterless polymer with adhesive properties and carbon particles, powder, fibers, flakes, granules and/or nanotubes. The adhesive polymer may be, for example, an acrylic polymer or a silicone polymer, or combination thereof. In some embodiments, the acrylic polymer or silicone polymer (and composites therefrom) may not be tacky, or may be minimally tacky, and yet may have adhesive properties in that the polymer may still aid in adhering the array to the skin. Herein, such materials may also be conductive adhesive materials. In some embodiments, the acrylic polymer or silicone polymer (or respective composites) may be available as acrylic- or silicone-based carbon-filled adhesive tapes. The adhesive may additionally include one or more conductive polymers (such as, for example, polyaniline (PANI), or poly(3,4-ethylenedioxythiophene) (PEDOT), or others known in the art). The conductive filler in the at least one layer of conductive adhesive material 316 or conductive material 318 may be non-metallic. In these embodiments, the conductive adhesive may have a thickness between 30 and 2,000 μm, such as, from 20 to 3,000 μm, or 30 to 400 μm.

In some embodiments, the transducer 300 may be constructed using a pre-formed 3-(or more) layer laminate comprising the conductive material 318, the anisotropic material layer 310, and the at least one layer of conductive adhesive material 316. In some embodiments, the at least one conductive adhesive material 316 and the conductive material 318 may both be conductive adhesive composites as described above, and the anisotropic material layer 310 may be a thin sheet of synthetic graphite such as pyrolytic graphite, as described above. In some embodiments, the at least one conductive adhesive material 316 and the conductive material 318 may be the same material or may be different. By way of example, in some embodiments, both the conductive adhesive material 316 and the conductive material 318 may comprise an acrylic polymer and a carbon powder filler; or both the conductive adhesive material 316 and the conductive material 318 may comprise an acrylic polymer and a carbon fiber filler. In some embodiments, the conductive adhesive material 316 may comprise an acrylic polymer and a carbon fiber filler, and the conductive material 318 may comprise an acrylic polymer and a carbon powder filler; or vice-versa. In some embodiments, one or both of the conductive adhesive material 316 and the conductive material 318 may be a hydrogel.

The substrate 304 may also include the hole 322 and may be aligned with the hole 322 through the anisotropic material layer 310, the conductive adhesive material 316, and the conductive material 318, such that the hole 322 extends through each layer located in the same position. The hole 322 may be in the center or substantially in the center of the transducer apparatus 300. In some embodiments, the hole 322 may be circular, oval, or ovoid shaped, or substantially circular, oval, or ovoid shaped. In some embodiments, the hole 322 may be at least 0.5 cm and at most 3.0 cm in diameter. For example, the hole 322 may be 0.5 cm, 3.0 cm, 3.5 cm, 2.0 cm, 2.5 cm, or 3.0 cm in diameter, or may range in diameter between any of these diameters. In some embodiments, the hole 322 may be at least 0.5% to at most 5.0% of the area of the anisotropic material layer 310. For example, the hole 322 may be 0.5%, 3.0%, 3.5%, 2.0%, 2.5%, 3.0%, 3.5%, 4.0%, 4.5%, or 5.0% of the area of the anisotropic material layer 310, or may range in diameter between any of these percentages. In some embodiments, the hole 322 may be at least 0.5% to at most 30%, or at most 20%, or at most 30%, or at most 40%, or at most 50%, of the area of the anisotropic material layer 310. In some embodiments, the hole 322 may be at least 0.5%, or at least 30%, or at least 20%, or at least 30%, or at least 40%, to at most 50%, of the area of the anisotropic material layer 310. In some embodiments, the hole in the substrate 304 may have a diameter smaller than the hole 322 of the anisotropic material layer 310. In some embodiments, the hole in the substrate 304 may have a diameter greater than the hole 322 of the anisotropic material layer 310. In some embodiments, the hole in the substrate 304 may match the size, shape, and location of the hole in the anisotropic material layer 310. In other embodiments, the substrate 304 may effectively cover the hole 322. In alternative embodiments, the substrate 304 may not cover the hole 322, but one or more layer of adhesive (e.g., conductive adhesive material 316 and/or the conductive material 318) may cover/fill the hole 322.

The transducer 300 may further include at least one foam material layer 324. The foam material layer 324 may be positioned between a front face 330 of the conductive adhesive material 316 and the subject's body. A back face 343 of the hole covering portion 342 of the foam material layer 324 may not be in contact with any other part or portion of the transducer apparatus 300. By being free of contact on the back face 343, the transducer apparatus 300 may be more pliable against a subject's body.

The foam material layer 324 may facilitate a comfortable surface against the subject's body. In some embodiments, a transducer 300 having the anisotropic material layer 310 may include the foam material layer 324 to provide a conformable material in contact with the subject's body. The foam material layer 324 may be in the same plane as the conductive adhesive layer 316 so that the electrode element(s) 302 may be placed as close as possible to the subject's skin, without the area of the electrode element 302 of the transducer apparatus 300 projecting past the foam material layer 324. For example, as shown in FIG. 3C, the right side 302R of the electrode 302 is to the left of the left side 324L of the foam material layer 324.

In some embodiments, the foam material layer 324 may be formed at least partially of a soft material with airy open space. In some embodiments, the foam material layer 324 may be formed of a non-porous material to improve sterilizability and cleanability. In some embodiments, the foam material layer 324 may comprise one or more of low-density polyethylene (LDPE), polyether olefin (polyether), silicone, polyurethane, or ethylene-vinyl acetate (EVA). In each case, the foam may be a closed-cell foam or an open-cell foam, or a combination thereof.

The transducer apparatus 300 may comprise an array of substantially flat electrode elements 302. The array of electrode elements 302 may or may not be capacitively coupled. The electrode elements 302 may be non-ceramic dielectric materials positioned over a plurality of flat conductors such as, for example, polymer films disposed over pads on a printed circuit board or over flat pieces of metal. In some embodiments, the electrode elements 302 are ceramic elements. In some embodiments, the electrode elements do not have a dielectric material.

In some embodiments, the dielectric material of the electrode elements 302 may have a dielectric constant ranging from 30 to 50,000. In some embodiments, the layer of dielectric material may comprise a high dielectric polymer material such as poly(vinylidene fluoride-trifluoroethylene-chlorotrifluoroethylene) and/or poly(vinylidene fluoride-trifluoroethylene-3-chlorofluoroethylene). Those two polymers are abbreviated herein as “Poly(VDF-TrFE-CTFE)” and “Poly(VDF-TrFE-CFE),” respectively. The dielectric constant of these materials may be on the order of 40. In some embodiments, the polymer layer may be poly(vinylidene fluoride-trifluoroethylene-chlorotrifluoroethylene-chlorofluoroethylene) or “Poly(VDF-TrFE-CTFE-CFE).”

In some embodiments, the layer of dielectric material of the electrode elements 302 may comprise a terpolymer comprising polymerized units of monomers such as VDF, TrFE, CFE and/or CTFE in any suitable molar ratio. Suitable terpolymers may include those, for example, having 30 to 80 mol % VDF, 5 to 60 mol % TrFE, with CFE and/or CTFE constituting the balance of the mol % of the terpolymer.

### Example Third Transducers

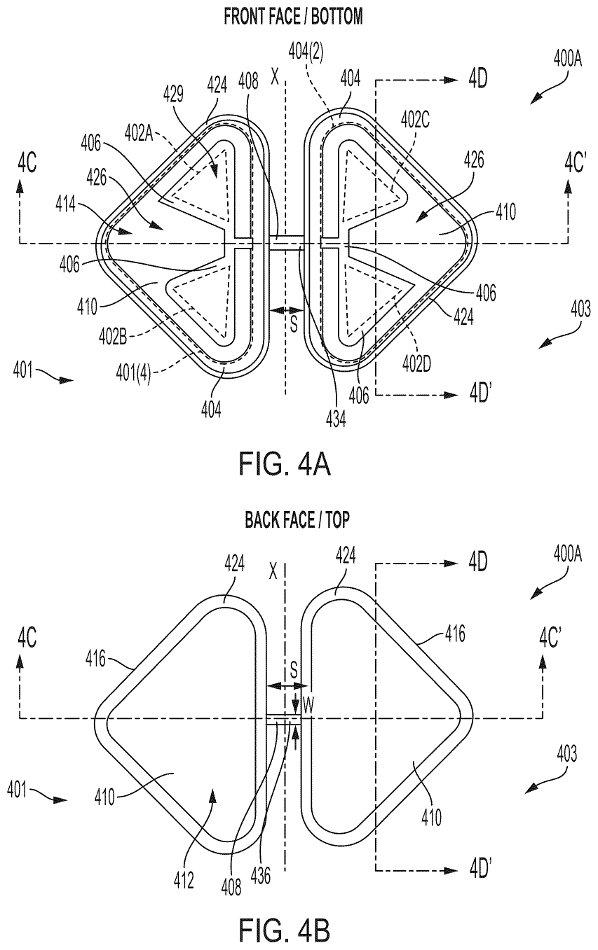

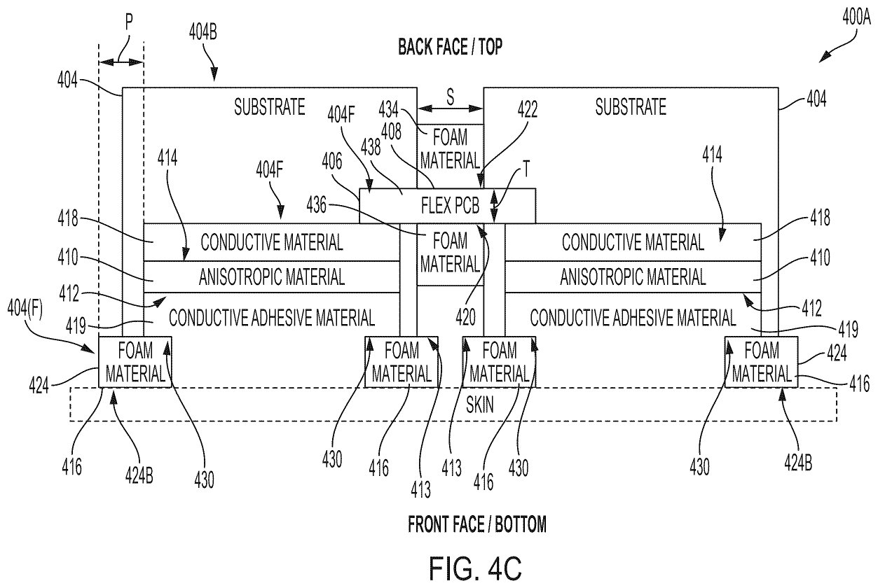

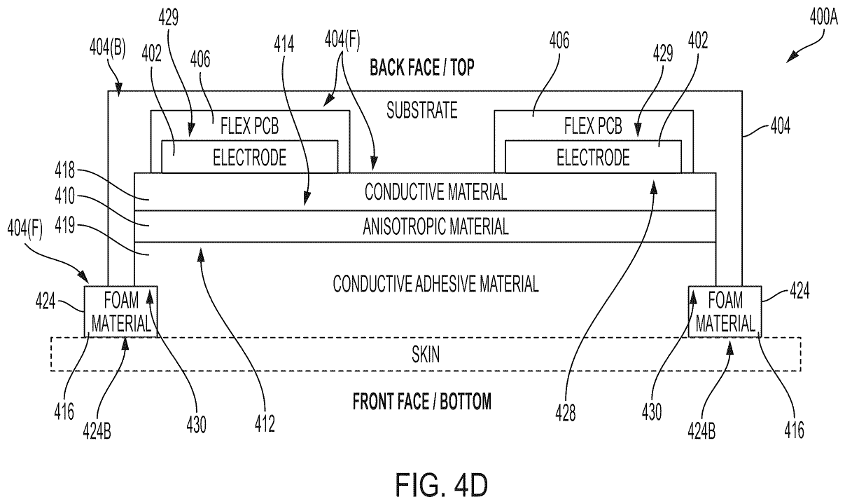

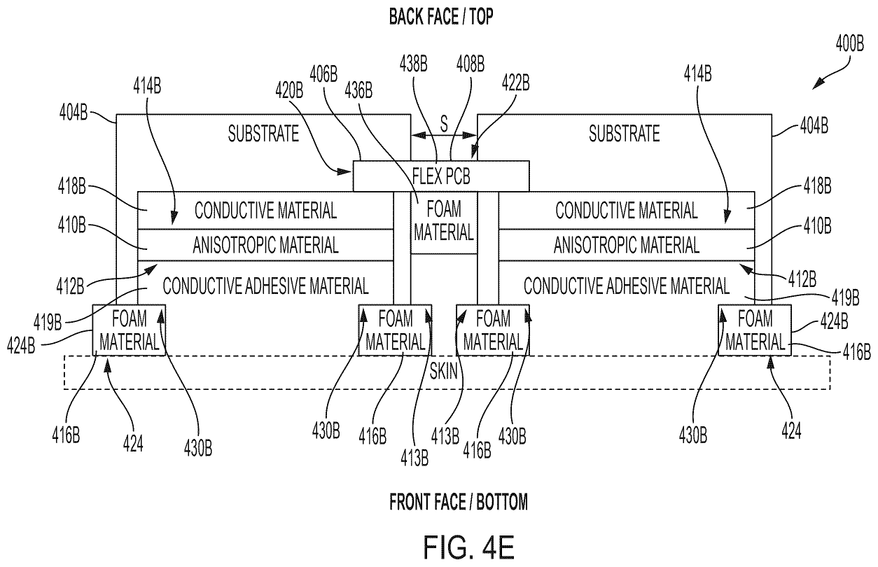

FIGS. 4A and 4B depict a top plan view (i.e., a back face plan view) and a bottom plan view (i.e., a front face view), respectively, of an example transducer apparatus 400A according to some embodiments. FIG. 4C depicts a cross-section view of the example transducer apparatus 400A of FIGS. 4A and 4B taken across section 4C-4C′ according to some embodiments. FIG. 4D depicts a cross-section view of the example transducer apparatus 400A of FIGS. 4A and 4B taken across section 4D-4D′ according to some embodiments.

As shown in FIGS. 4A and 4B, the transducer apparatus 400A may include a first electrode subassembly (or first transducer subassembly) 401 and a second electrode subassembly (or second transducer subassembly) 403. The first electrode subassembly 401 and the second electrode subassembly 403 may have shapes that are symmetrical along the sagittal plane X and include the same components. Thus the following description of the components apply to both the first and the second electrode subassemblies 401, 403.

Each of the first and the second electrode subassemblies 401, 403 may include an array of at least one electrode 402 (e.g., 402A, 402B, 402C, 402D), a flexible printed circuit board (PCB) 406 electrically connecting the electrode elements 402, an anisotropic material layer 410 electrically coupled to the array of at least one electrode 402, and a substrate 404 for holding the array of at least one electrode and the anisotropic material layer against the subject's body. The first and the second electrode subassemblies 401, 403 may be connected with a flexible electrical connector 408, where the flexible electrical connector 408 separates the first and the second electrode subassemblies 401, 403. In FIG. 4A, the electrode elements 402A, 402B, 402C, and 402D are shown in dashed outline, as they are located between the flexible PCB 406 and the anisotropic material layer 410. In FIG. 4A, although the substrate 404 covers the back sides of the first and the second electrode subassemblies 401, 403, the substrate 404 is shown as a cut-away in a partial view by the dashed lines 401(4) and 404(2) so as to illustrate the components beneath the substrate 404. As illustrated in FIGS. 4C and 4D, the substrate 404 covers the back sides of first and second electrode subassemblies 401, 403.

When viewed in a direction perpendicular to and toward the back face of the subassemblies 401, 403 (as in FIG. 4A), the first and second electrode subassemblies 401, 403 may have a same shape, or may have a mirror-image shape of one another. In some embodiments, each of the first and second electrode subassemblies 401, 403 may be triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped, or each subassembly has an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature. For example, in FIG. 4A, the first and second electrode subassemblies 401, 403 may be triangular or rounded triangular shaped, and may be a mirror-image shape of one another.

In some embodiments, the first and the second electrode subassemblies 401, 403 may be the same size, have the same shape, have the same dimensions, and have the same surface area. In some embodiments, the first and the second electrode subassemblies 401, 403 may be different sizes and/or shapes. For example, one of the electrode subassemblies may have a surface area at least 25% and at most 50% larger (including any percentage therebetween) than the surface area of the other electrode subassembly.

Each of the first and the second electrode subassemblies 401, 403 may include an array of at least one electrode 402. In some embodiments, each of the first and the second electrode subassemblies 401, 403 has at least two electrode elements 402. In some embodiments, each of the first and the second electrode subassemblies 401, 403 has only one electrode element 402. In some embodiments, the subassemblies 401, 403 may have the same number of electrode elements 402. In some embodiments, the subassemblies 401, 403 may have a different number of electrode elements 402.

The electrodes elements 402 of the first and the second electrode subassemblies 401, 403 may be adapted to administer TTFields therapy to a subject. The electrodes elements 402 may be electrically conductive and may be substantially flat. The electrode elements 402 may or may not be capacitively coupled. The electrodes elements 402 may be metal, metal alloy, layered metal, laminated conductive material, or may be ceramic or non-ceramic dielectric material positioned over a flat conductor (e.g., polymer film disposed on flat metal). In some embodiments, the electrode elements do not have a dielectric material.

In some embodiments, the dielectric material of the electrode elements 402 may have a dielectric constant ranging from 10 to 50,000. In some embodiments, the layer of dielectric material may comprise a high dielectric polymer material such as poly(vinylidene fluoride-trifluoroethylene-chlorotrifluoroethylene) and/or poly(vinylidene fluoride-trifluoroethylene-4-chlorofluoroethylene). Those two polymers are abbreviated herein as “Poly(VDF-TrFE-CTFE)” and “Poly(VDF-TrFE-CFE),” respectively. The dielectric constant of these materials may be on the order of 40. In some embodiments, the polymer layer may be poly(vinylidene fluoride-trifluoroethylene-chlorotrifluoroethylene-chlorofluoroethylene) or “Poly(VDF-TrFE-CTFE-CFE).”

In some embodiments, the layer of dielectric material of the electrode elements 402 may comprise a terpolymer comprising polymerized units of monomers such as VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer.

The first and the second electrode subassemblies 401, 403 may include the same number or different numbers of electrodes elements 402. In some embodiments, the first and second electrode subassemblies 401, 403 may each (independently) include one, two, three, four, five, six, seven, eight, nine, ten, eleven, twelve, thirteen, fourteen, fifteen, sixteen, seventeen, eighteen, nineteen, twenty, or more electrodes elements 402. For example, in FIG. 4A, the first and the second electrode subassemblies 401, 403 are depicted as having two electrodes elements each. The first electrode subassembly 401 may include two electrodes 402A, 402B, and the second electrode subassembly 403 may include two electrodes 402C, 402D.

In some embodiments, the electrodes elements 402 may have a same shape. In some embodiments, when viewed in a direction perpendicular to and toward the back face of the subassemblies, the electrodes element 402 may be triangular shaped, substantially triangular shaped, rounded triangular shaped, substantially rounded triangular shaped, kidney bean or jelly bean shaped, substantially kidney bean or jelly bean shaped, ovoid shaped, substantially ovoid shaped, oval shaped, or substantially oval shaped; or each subassembly may have an asymmetric oval, ovaloid, ovoid, or ovate shape or a stretched asymmetric oval, ovaloid, ovoid, or ovate shape with a first end and a second end, the first end having a first radius of curvature, the second end being opposite the first end and having a second radius of curvature, the first radius of curvature being greater than the second radius of curvature. For example, in FIG. 4A, the first and the second electrode subassemblies 401, 403 are depicted as having two triangular shaped (or rounded triangular shaped) electrodes elements each. The first electrode subassembly 401 includes two triangular or rounded triangular shaped electrodes 402A, 402B, and the second electrode subassembly 403 includes two triangular or rounded triangular shaped electrodes 402C, 402D.

The flexible PCB 406 may electrically couple together the electrode elements 402. In some embodiments, the flexible PCB 406 may cover the back faces 429 (FIGS. 4A, 4D) of the electrode elements 402. An edge of the electrode element 402 may be offset inwardly from an edge of the flexible PCB 406. In some embodiments, when viewed in a direction perpendicular to and toward the back face of the electrode subassemblies 401, 403, the flexible PCB 406 may cover up to and including 45%, 20%, 30%, 40%, 60%, 80%, or any percentage therebetween of a back face 414 (FIGS. 4A, 4C, 4D) of the anisotropic material layer 410. In some embodiments, the flexible PCB 406 may not function as an electrode. The flexible PCB 406 may be a non-adhesive region. In some embodiments, the flexible PCB 406 may comprise a polyimide film, such as, for example, Kapton® (from DuPont, Wilmington, DE, USA) and conductive traces (for example, and without limitation, copper traces or traces of conductive ink, etc.).

In some embodiments, each subassembly 401, 403, may have one or more temperature sensors (such as, for example thermistors) (not shown) associated with some or all electrode elements 402 of the respective array.

The flexible electrical connector 408 may extend between and electrically couple the first and the second electrode subassemblies 401, 403. In some embodiments, the flexible electrical connector 408 may include a portion 438 of the flexible PCB 406 to electrically connect the first and the second electrode subassemblies 401, 403 (FIG. 4C). The portion 438 of the flexible PCB 406 may be part of neither the first nor the second electrode subassemblies 401, 403. The flexible electrical connector 408 may electrically connect the array of at least one electrode 402 of the first electrode subassembly 401, and the array of at least one electrode 402 of the second electrode subassembly 403.

The flexible electrical connector 408 may include a front face 420 facing the subject's body and a back face 422 opposite the front face. The front face 420 of the flexible electrical connector 408 may be free of adhesive material (e.g., as depicted in FIG. 4C). When the subassemblies 401, 403 are viewed in cross-section, the flexible electrical connector 408 may be adapted not to be adhesively held to the subject's body.

The flexible electrical connector 408 may further include a foam material layer, such as a back foam material layer (or top foam material layer) 434 on the back face 422 of the flexible electrical connector 408 and/or a front foam material layer (or bottom foam material layer) 436 on the front face 420 of the flexible electrical connector 408. In some embodiments, the flexible electrical connector 408 may be a layered component including one or more of the back foam material layer 434, the portion 438 of the flexible PCB 406, and the front foam material layer 436. In some embodiments, the flexible electrical connector 408 may be or may include a plastic coated wire to electrically connect the first and the second electrode subassemblies 401, 403. In some embodiments, one or more wedges may reside between the flexible electrical connector 408 and the subject's body, and the one or more wedges may comprise foam material.

In some embodiments, when viewed in a direction perpendicular to and toward the back face of the subassemblies 401, 403 (e.g., as depicted in FIG. 4A), the flexible electrical connector 408 may have a substantially linear shape. In some embodiments, when viewed in a direction perpendicular to and toward the back face of the subassemblies 401, 403, the flexible electrical connector 408 may have different shape, such as, for example: a U-shape, substantially U-shape, or rounded V-shape; a shape with two or more bends, such as an undulating shape, serpentine shape, wavelike shape, zig-zag shape; a shape with a combination of concave and convex portions; or a spiral shape.

In some embodiments, the flexible electrical connector 408 may be adapted to be deformed to a substantially concave shape away from the subject's body. In some embodiments, when the subassemblies are viewed in cross-section, the flexible electrical connector 408 may be adapted to deform from a substantially planar shape to a U-shape, a substantially U-shape, or a rounded V-shape. In some embodiments, when the subassemblies 401, 403 are viewed in cross-section, the flexible electrical connector 408 may be adapted to be deformed to have a void between the flexible electrical connector 408 and the subject's body.

The flexible electrical connector 408 may provide an adjustable distance between the first and the second electrode subassemblies 401, 403. The flexible electrical connector 408 may be bent such that the first and the second electrode subassemblies 401, 403 are close together, but not touching (see, e.g., FIGS. 3A and 3B). When bent, the flexible electrical connector 408 may form a tent-like shape where the apex points away from the subject, and thus, the flexible electrical connector 408 may deform to a substantially concave shape away from the subject's body. The “tenting” of the flexible electrical connector 408 may allow for improved heat dissipation. The flexible electrical connector 408 may be pulled taught such that the first and the second electrode subassemblies 401, 403 are at a maximum distance, measured along the surface of the transducer apparatus 400A.

The flexible electrical connector 408 may have a length between the first and the second electrode subassemblies 401, 403 of at least 0.05 cm, or at least 0.4 cm, and not more than 5.00 cm. When viewed in a direction perpendicular to and toward the back face of the subassemblies 401, 403 (e.g., as depicted in FIG. 4A), the flexible electrical connector 408 may provide a distance S between the first and the second electrode subassemblies 401, 403. The portion 438 of the flexible PCB 406 may connect the first and the second electrode subassemblies 401, 403 across the distance S. Due to the flexible electrical connector 408 being flexible, the distance S between the first and the second electrode subassemblies 401, 403 may vary as the subject moves. The flexible electrical connector 408 may provide a maximum distance S between the first and the second electrode subassemblies 401, 403 when the flexible electrical connector 408 is planar.

In some embodiments, the distance S may be subject-specific. For example, the size of the subject may be considered when determining the subject-specific distance S, and an appropriately-sized transducer apparatus may be selected from a variety of sizes (e.g., small, medium, and large).

In some embodiments, the flexible electrical connector 408 may have a thickness T (between the back face 422 and the front face 420, as depicted in FIG. 4C) and a width W (as depicted in FIG. 4B) that is greater than the thickness. For example, the width of the flexible electrical connector 408 may be at least 5 times greater than the thickness of the flexible electrical connector 408. In some embodiments, the width of the flexible electrical connector 408 may be at least 0.5 centimeter, or at least 1 centimeter, or at least 2 centimeters, or at least 5 centimeters. It is contemplated that the flexible electrical connector 408 having such a width may inhibit undesired twisting of the flexible electrical connector 408 that could affect the spacing between the first and the second electrode subassemblies 401, 403.

In some embodiments, the flexible electrical connector 408 may be attached to the first and the second electrode subassemblies 401, 403 to permit angular pivoting (or angular rotating). For example, the flexible electrical connector 408 may have a sufficient width to permit angular pivoting (or angular rotating) of the first and the second electrode subassemblies 401, 403 about an axis that extends out of the page in FIG. 4A.

The transducer apparatus 400A may include one or more blank spaces 426 (or void spaces), which do not overlap with any of the electrode elements 402 and include the anisotropic material layer 410. Upon a rotational or translational shift of the subassemblies 401, 403, while (approximately) remaining in the preferred location with respect to treating a target in/on the subject's body, at least part of one or more of the blank spaces 426 may provide a relief region for the subject since no electrode elements 402 are in the blank spaces 426.

The transducer apparatus 400A may include the anisotropic material layer 410 directly or indirectly electrically coupled to the plurality of electrodes 402 and located on a front face 428 (FIG. 4D) of the electrodes 402 configured to face the subject's body. The anisotropic material layer 410 may take any of the forms and include any of the features described in further detail herein with reference to the anisotropic material layer.

The anisotropic material layer 410 may be disposed over (and in front of) the plurality of electrode elements 402 such that the anisotropic material layer 410 covers the electrode elements 402 and, optionally, the at least one blank space 426. In some embodiments, the anisotropic material layer 410 may not extend outward all the way to the edge of the substrate layer 404.

The transducer apparatus 400A may further include a foam material layer 424 directly or indirectly coupled to the anisotropic material layer 410, located on a front face 412 of the anisotropic material layer 410, and configured to contact the subject's body. In some embodiments, a layer of conductive adhesive material or conductive hydrogel may be located on the front face 412 of the anisotropic material layer 410 and may act as a biocompatible skin-contact layer. A front face 424B of the foam material layer 424 may include an adhesive to aid in securing the transducer apparatus 400A to the subject's skin. When viewed in a direction perpendicular to and toward the front face of the subassemblies 401, 403 (e.g., as depicted in FIG. 4B), the foam material layer 424 may have an empty center such that the foam material layer 424 frames the anisotropic material layer 410. In some embodiments, the foam material layer 424 may be a continuous layer that covers some or most of the front face 412 of the anisotropic material layer 410 but does not cover all of the front face 412 of the anisotropic material layer 410. In some embodiments, the anisotropic material layer 410 may not extend outward all the way to the edge of the foam material layer 424. The foam material layer 424 may cover at most 5%, or at most 10%, or at most 20%, or at most 30%, or at most 40%, or at most 50%, or any percentages therebetween, of the anisotropic material layer 410.

When viewed in a direction perpendicular to and toward the front face 412 of the anisotropic material layer 410 of each subassembly 401, 403, the foam material layer 424 may include a perimeter 416 covering a perimeter of the anisotropic material layer 410 (FIGS. 4B, 4C, 4D). In some embodiments, the perimeter 416 of the foam material layer 424 may cover up to the edge of the perimeter of the anisotropic material layer 410. In some embodiments, the perimeter 416 of the foam material layer 424 may be larger than the perimeter of the anisotropic material layer 410. The perimeter 416 of the foam material layer 424 may have substantially the same shape as the perimeter of the anisotropic material layer 410. In some embodiments, an offset distance P between the perimeter 416 of the foam material layer 424 and the perimeter of the anisotropic material layer 410 (e.g., as depicted in FIG. 4C) may be the same about the perimeter of the subassemblies 401, 403. Alternatively, the offset distance P between the perimeter of the anisotropic material layer 410 and the perimeter portion 416 of the foam material layer 424 (as in FIG. 4C) may vary about the perimeter of the subassemblies 401, 403.

The foam material layer 424 may facilitate a comfortable surface against the subject's body. In some embodiments, a transducer 400A having the anisotropic material layer 410 may include the foam material layer 424 to provide a conformable material in contact with the subject's body. The foam material layer 424 may be in the same plane as the conductive adhesive layer 419 so that the electrode element 402 may be placed as close as possible to the subject's skin, without the area of the front face 428 of the electrode element 402 of the transducer apparatus 400A projecting past the foam material layer 424. A back face 413 of the foam material layer 424 may not be in contact with the flexible electrical connector 408.

In some embodiments, the transducer 400A may include a foam material layer 436 disposed on the front face 420 of the flexible electrical connector 408 and/or another foam material layer 434 disposed on the back face 422 of the flexible electrical connector 408 (e.g., as depicted in FIG. 4C). In some embodiments, the foam material layer 424 may cover the front face 420 of the portion 438 of the flexible PCB 406. The foam material layer 436 on the front face 420 of the flexible electrical connector 408 may be a portion of the foam material layer 424 or may be separate from the foam material layer 424. In some embodiments, the foam material layer 436 and the foam material layer 424 may be separated from each other such that they form neither a contiguous nor a unitary body. As such, when the flexible electrical connector 408 is deformed in a concave shape or “tented,” a back face 413 of the foam material layer 424 is not in contact with the flexible electrical connector 408. The transducer apparatus 400A may be more pliable against a subject's body when the flexible electrical connector 408 is free of contact with the back face 413 of the foam material layer 424.

The foam material layers 434, 436 may provide a comfortable surface against the subject's body. During certain treatments, the electrode subassemblies 401, 403 may need to be spaced apart at the maximum distance permitted by the length of the flexible electrical connector 408. As such, the flexible electrical connector 408 will be substantially flat and in contact or close to contact with the subject's body. The foam material layer 434, 436 may be included to provide a comfortable material in contact with the subject's body.

In some embodiments, the foam material layers 424, 434, and 436 may be at least 2 mm and at most 40 mm thick, or at least 5 mm and at most 40 mm thick, or at least 8 mm and at most 40 mm thick. In some embodiments, the foam material layer 424, 434, 436 may be formed at least partially of a soft material with airy open space. In some embodiments, the foam material layer 424, 434, 436 may be formed of a non-porous material to improve sterilizability and cleanability. In some embodiments, the foam material layer 424, 434, 436 may comprise one or more of low-density polyethylene (LDPE), silicone, polyurethane, or ethylene-vinyl acetate (EVA), each of which may be an open-cell, closed-cell, or partially open/closed-cell foam. In some embodiments, a closed-cell foam may be preferred. In some embodiments, the foam may be an adhesive coated foam. In some embodiments, the foam may be a 3M™ Tegaderm™ product (3M, Saint Paul, MN, USA), which may include adhesive on one side facing the subject's body and facing away from the substrate 404.

The substrate 404 may be configured for attaching the transducer 400A to a subject's body and may be configured to secure the various components of the transducer 400A. The substrate 404 may hold the array of at least one electrode 402 and the anisotropic material layer 410 against the subject's body. The substrate 404 of each subassembly 401, 403 may include a front face 404(F) facing the subject's body and a back face 404(B) opposite the front face 404(F) (e.g., as depicted in FIGS. 4C and 4D). The front face 404(F) of the substrate 404 may face the array of at least one electrode 402 and the anisotropic material layer 410. The substrate 404 of each subassembly 401, 403 may have an outer perimeter extending beyond an outer edge of the respective anisotropic material layer 410 of each subassembly. The substrate 404 of each subassembly 401, 403 may be contoured to match a shape of the outer edge of each respective anisotropic material layer 410. The substrate 404 of each subassembly 401, 403 may have an outer perimeter extending beyond the outer edge of all other components of each subassembly, such that an adhesive on the front face 404(F) of the substrate 404 adheres the substrate 404 to the subject's body, thereby holding each subassembly against the subject's body. The substrates 404 of the first and second electrode subassemblies 401, 403 may be separated by the flexible electrical connector 408. Accordingly, in some embodiments, the substrates 404 of the first and second electrode subassemblies 401, 403 are distinct and not a unitary body.

Each of the subassemblies 401, 403 may have separated portions of the substrate 404. In some embodiments, the flexible electrical connector 408 may not include a portion of the substrate 404. In some embodiments, the substrate 404 may not directly contact the subject's skin due to the foam material layer 424. Suitable materials for the substrate 404 may include, for example, tape, bandage, cloth, nonwoven fabric, foam, flexible plastic, and/or a conductive medical gel. The transducer 400A may be affixed to the subject's body via the substrate 404 (e.g., via an adhesive layer and/or a conductive medical gel). The substrate 404 may be an adhesive bandage. The substrate 404 may aid in securing a cable (not shown) connected to the flexible PCB 406 for providing signals to the flexible PCB 406 for generating TTFields with the transducer apparatus 400A.

As discussed above, the transducer apparatus 400A further may include the anisotropic material layer 410. As shown, the anisotropic material layer 410 may have a front face 412 and a back face 414, wherein the back face 414 faces the array of electrode elements 402 (e.g., as depicted in FIG. 4D). The anisotropic material layer 410 may have anisotropic thermal properties and/or anisotropic electrical properties. If the anisotropic material layer 410 has anisotropic thermal properties (for example, greater thermal conductivity in the plane of the layer than through the plane of the layer), then the layer may spread the heat out more evenly over a larger surface area. If the anisotropic material layer 410 has anisotropic electrical properties (for example, greater electrical conductivity in the plane of the layer than through the plane of the layer), then the layer may spread the current out more evenly over a larger surface area. In each case, the temperature of the hot spots may be lowered and the temperature of the cooler regions may be raised when a given AC voltage is applied to the array of electrode elements. Accordingly, the current may be increased (thereby increasing the therapeutic effect) without exceeding the safety temperature threshold at any point on the subject's skin.

In some embodiments, the anisotropic material layer 410 may be anisotropic with respect to electrical conductivity properties. In some embodiments, the anisotropic material layer 410 may be anisotropic with respect to thermal conductivity properties. In some embodiments, the anisotropic material layer 410 may be anisotropic with respect to both electrical conductivity properties and thermal conductivity properties.

The anisotropic thermal properties may include directional thermal properties. Specifically, the anisotropic material layer 410 may have a first thermal conductivity in a direction that is perpendicular to its front face (skin-facing surface) 412 that may be different from a thermal conductivity of the anisotropic material layer 410 in directions that are parallel to the front face 412. For example, the thermal conductivity of the anisotropic material layer 410 in directions parallel to the front face 412 may be more than two times higher than the first thermal conductivity. In some embodiments, the thermal conductivity in the parallel directions may be more than ten times higher than the first thermal conductivity. For example, the thermal conductivity of the sheet in directions that are parallel to the front face 412 may be more than: 1.5 times, 2 times, 3 times, 5 times, 10 times, 20 times, 100 times, 200 times, or even more than 1,000 times higher than the first thermal conductivity.

The anisotropic electrical properties may include directional electrical properties. Specifically, the anisotropic material layer 410 may have a first electrical conductivity (or, conversely, resistance) in a direction that is perpendicular to its front face 412 that may be different from an electrical conductivity (or resistance) of the anisotropic material layer 410 in directions that are parallel to the front face 412. For example, the resistance of the anisotropic material layer 410 in directions parallel to the front face 412 may be less than the first resistance. In some embodiments, the resistance in the parallel directions may be less than half of the first resistance or less than 40% of the first resistance. For example, the resistance of the anisotropic material layer 410 in directions that are parallel to the front face 412 may be less than: 75%, 50%, 40%, 30%, 20%, 10%, 5%, 1%, 0.5%, or even less than 0.1% of the first resistance.

In some embodiments (e.g., when the anisotropic material layer 410 is a sheet of pyrolytic graphite), the anisotropic material layer 410 may have both anisotropic electrical properties and anisotropic thermal properties.

The anisotropic material layer 410 may comprise graphite (e.g., a sheet of graphite or a graphite sheet). Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2040A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). In alternative embodiments, conductive anisotropic materials other than graphite may be used instead of graphite.

In some embodiments, the anisotropic material layer 410 may be a sheet of pyrolytic graphite. Thermal conductivity of pyrolytic graphite sheets in directions that are parallel to the front face 412 of those sheets may be typically more than 50 times higher than the thermal conductivity of those sheets in directions that are perpendicular to the front face 412. Electrical resistivity of pyrolytic graphite sheets in directions that are parallel to the front face 412 of those sheets may be typically less than 2% of the electrical resistivity of those sheets in directions that are perpendicular to the front face 412.

The transducer 400A may further include at least one layer of conductive adhesive material 419 and/or hydrogel disposed on a front facing side 412 of the anisotropic material layer 410. In some embodiments, the at least one layer of conductive adhesive material 419 may be disposed on the front face 412 of the anisotropic material layer 410. The at least one layer of conductive adhesive material 419 may have a biocompatible front surface. In some embodiments, there may be only a single layer of conductive adhesive material 419, and that single layer (the front layer) may be biocompatible. In some embodiments, there may be more than one layer of conductive adhesive material 419, in which case only the front layer may be biocompatible, or the front layer and one or more other layers may be biocompatible. In some embodiments, the front layer of conductive adhesive material 419 may be configured to ensure good electrical contact between the device and the body. In some embodiments, the front layer of conductive adhesive material 419 may be configured to ensure good adhesion between the anisotropic material layer 410 and the foam material layer 424. In some embodiments, the front layer of conductive adhesive material 419 may cover the entire front face 412 of the anisotropic material layer 410. The front layer of conductive adhesive material 419 may be the same size or larger than the anisotropic material layer 410. In some embodiments, a front face of conductive adhesive material 419 may comprise hydrogel. In these embodiments, the hydrogel may have a thickness between 50 μm and 2,000 μm. In some embodiments, the front layer of conductive adhesive material 419 may comprise a conductive adhesive composite as further disclosed herein.

The transducer 400A may further include at least one layer of conductive material 418, such as a conductive adhesive material or hydrogel, disposed on a back facing side 414 of the anisotropic material layer 410. The conductive material 418 may be positioned between the array of electrode elements 402 and the back face 414 of the anisotropic material layer 410 facing the array (e.g., as depicted in FIG. 4D). The conductive material 418 may facilitate the electrical contact between the array of electrode elements 402 and the back face 414 of the anisotropic material layer 410. In some embodiments, the conductive material 418 may be a layer of conductive adhesive material. In some embodiments, the conductive material 418 may be a layer of hydrogel. In other embodiments, a different conductive material (e.g., conductive grease, conductive adhesives, conductive tape, etc.) may be used. For example, the conductive material 418 may comprise a conductive adhesive composite as further disclosed herein.

In some embodiments, the flexible PCB 406 may be positioned between a back face of the conductive material 418 and the substrate 404. In some embodiments, the electrode elements 402 may be positioned between the back face of the conductive material 418 and the substrate 404. In some embodiments, the front faces of the electrode elements 402 may be in direct contact with the back face of the conductive adhesive 418. In some embodiments, the foam material layer 424 may be positioned between a front face 430 of the conductive adhesive material 419 and the subject's body.

In some embodiments, the at least one layer of conductive adhesive material 419 and/or the layer of conductive material 418 may be a single layer of non-hydrogel conductive adhesive such as the developmental product FLX068983-FLEXcon® OMNI-WAVE™ TT 200 BLACK H-502 450 POLY H-9 41PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Non-hydrogel conductive adhesives may comprise a waterless polymer with adhesive properties and carbon particles, powder, fibers, flakes, granules and/or nanotubes. The adhesive polymer may be, for example, an acrylic polymer or a silicone polymer, or combination thereof. In some embodiments, the acrylic polymer or silicone polymer (and composites therefrom) may not be tacky, or may be minimally tacky, and yet may have adhesive properties in that the polymer may still aid in adhering the array to the skin. Herein, such materials may also be conductive adhesive materials. In some embodiments, the acrylic polymer or silicone polymer (or respective composites) may be available as acrylic- or silicone-based carbon-filled adhesive tapes. The adhesive may additionally include one or more conductive polymers (such as, for example, polyaniline (PANI), or poly(3,4-ethylenedioxythiophene) (PEDOT), or others known in the art). The conductive filler in the at least one layer of conductive adhesive material 419 or conductive material 418 may be non-metallic. In these embodiments, the conductive adhesive may have a thickness between 20 μm and 4,000 μm, such as between 40 μm and 2,000 μm, or such as between 30 μm and 400 μm.

In some embodiments, the transducer 400A may be constructed using a pre-formed 3-(or more) layer laminate comprising the conductive material 418, the anisotropic material layer 410, and the at least one layer of conductive adhesive material 419. In some embodiments, the at least one conductive adhesive material 419 and the conductive material 418 are both conductive adhesive composites as described above, and the anisotropic material layer 410 may be a thin sheet of synthetic graphite such as pyrolytic graphite, as described above. In some embodiments, the at least one conductive adhesive material 419 and the conductive material 418 may be the same material or may be different. By way of example, in some embodiments, both the conductive adhesive material 419 and the conductive material 418 may comprise an acrylic polymer and a carbon powder filler; or both the conductive adhesive material 419 and the conductive material 418 may comprise an acrylic polymer and a carbon fiber filler. In some embodiments, the conductive adhesive material 419 may comprise an acrylic polymer and a carbon fiber filler, and the conductive material 418 may comprise an acrylic polymer and a carbon powder filler; or vice-versa. In some embodiments, one or both of the conductive adhesive material 419 and the conductive material 418 may be a hydrogel.

FIG. 4E depicts a cross-section view of an example transducer apparatus 400B according to some embodiments. The transducer apparatus 400B in FIG. 4E may be the same as the transducer apparatus 400A in FIG. 4C, except for the flexible electrical connector 408B. The parts in FIG. 4E use the same reference numbering as the parts in FIG. 4C, except for the added suffix of “B”. In FIG. 4E, the flexible electrical connector 408B includes foam material layer 436B disposed on the front surface 420B and does not include a foam material layer disposed on the back surface 422B. The foam material layer 436B may facilitate a comfortable surface against the subject's skin. The flexible electrical connector 408B may have greater flexibility and ability to concave by including only one foam material layer. As shown, the substrate may not be continuous across the flexible electrical connector 408B, but in some embodiments, the substrate may be continuous across the flexible electrical connector 408B.

### Example Fourth Transducers

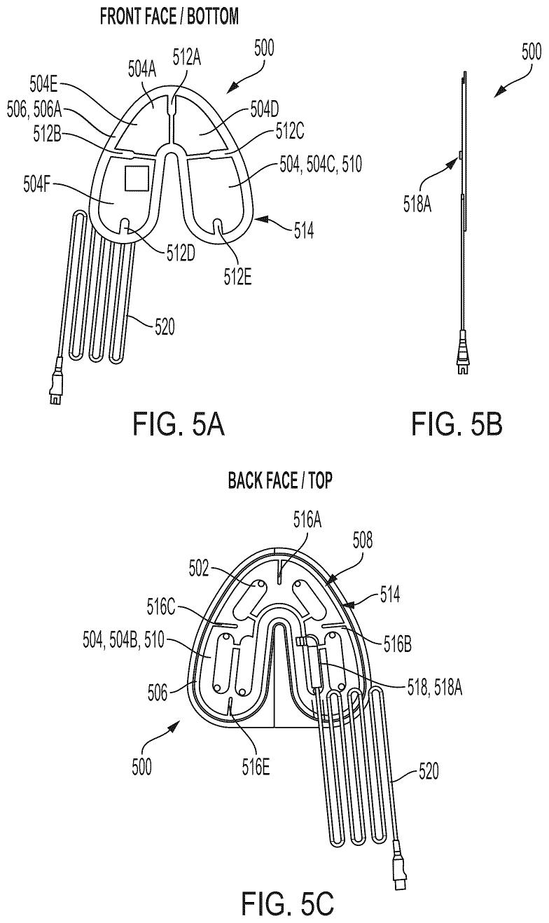

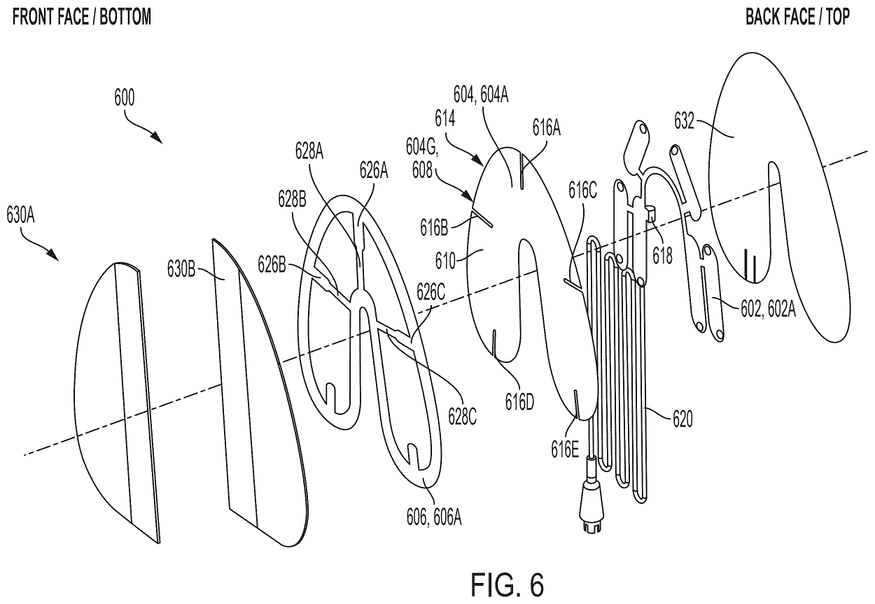

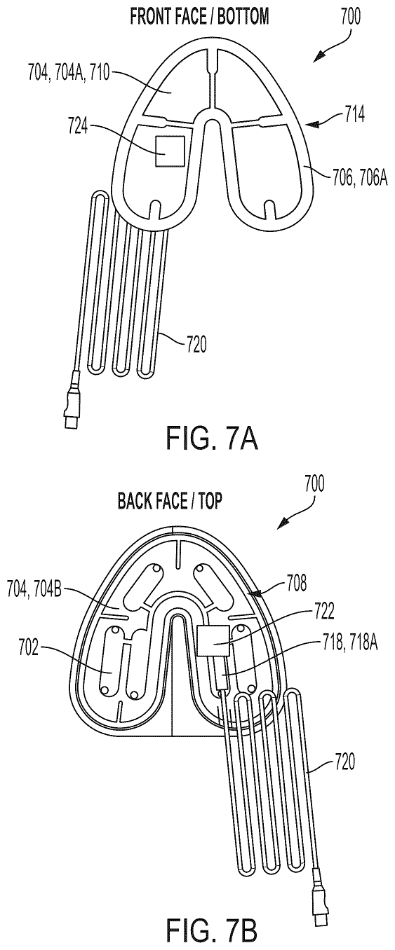

FIGS. 5A to 5C depict a bottom (front) plan view, a right plan view, and a top (back) plan view, respectively, of an example transducer apparatus 500 according to some embodiments. FIG. 6 depicts an exploded view of an example transducer apparatus 600 according to some embodiments. FIGS. 7A and 7B depict a bottom (front) plan view and a top (back) plan view, respectively, of an example transducer apparatus 700 according to some embodiments.

In FIGS. 5A-7B, the transducer 500, 600, 700 may include an array of one or more electrode elements 502, 602, 702, and an anisotropic material layer 504, 604, 704 coupled to the array of at least one electrode element 502, 602, 702. The array of electrode elements 502, 602, 702 may have a front face 502A, 602A (502A not shown in FIG. 5A-C), and the anisotropic material layer 504, 604, 704 may be located on a front side of the front face 502A, 602A of the array 502, 602, 602. As shown in FIGS. 5A and 5C, the anisotropic material layer 504, 604, 704 may have a front face 504A, 604A, 704A and a back face 504B, 704B, with the back face 504B, 704B facing the array of electrode elements 502, 602, 702.

In FIGS. 5A-7B, the transducers 500, 600, 700 may comprise an array of electrode elements 502, 602, 702 having one or more electrodes connected with wiring (e.g. a flexible printed circuit board (PCB)) (such as, for example, one or more electrodes 402 and a flexible PCB 406 electrically connecting the one or more electrodes 402). For each figure, the array of electrode elements 502, 602, 702 may be capacitively coupled or coupled via conductive wiring.

The anisotropic material layer 504, 604, 704 may be any conductive layer having different thermal and/or electrical conductivities in a direction perpendicular to the front face 504A, 604A, 704A of the anisotropic material layer 504, 604, 704 than in directions that are parallel to the front face 504A, 604A, 704A. The anisotropic material layer 504, 604, 704 may be anisotropic with respect to electrical conductivity properties, anisotropic with respect to thermal properties, or both. This allows the anisotropic material layer 504, 604, 704 to spread out current and/or heat over a larger surface area. In each case, the temperature of hot spots may be lowered and the temperature of cooler regions may be raised when a given AC voltage is applied to the array of electrode elements. Accordingly, the current can be increased without exceeding a safety temperature threshold at any point on the subject's skin. The anisotropic material layer 504, 604, 704 may be a sheet of graphite, such as a sheet of synthetic graphite. The anisotropic material layer 504, 604, 704 may be a sheet of pyrolytic graphite, graphitized polymer film, a graphite foil made from compressed high purity exfoliated mineral graphite, or some other material. Other details regarding the anisotropic material layer 504, 604, 704 and properties thereof are described in U.S. Patent Application Publication No. 2023/0037806 A1, Wasserman et al., Feb. 9, 2023, which is hereby incorporated herein by reference in its entirety.

The transducer 500, 600, 700 may further include a flexible layer 506, 606, 706 (hereinafter, “flexible layer”) which may be at least partially disposed on the front facing side 504A, 604A, 704A of the anisotropic material layer 504, 604, 704, optionally the flexible layer 506, 606, 706 may be disposed on the front face 504A, 604A, 704A of the anisotropic material layer 504, 604, 704, and may further be disposed over at least a portion of an outer perimeter 514, 614, 714 of the anisotropic material layer 504, 604, 704. In this way, the flexible layer 506, 606, 706 may define a seal between the anisotropic material layer 504, 604, 704 and a subject's body. The flexible layer 506, 606, 706 may be any suitable thickness, for example and not limitation 0.1 mm, 0.5 mm, 1.0 mm, 1.5 mm, 2 mm, or 5 mm. Furthermore, the flexible layer 506, 606, 706 may include adhesive (such as those described elsewhere herein). In some embodiments, the flexible layer 506, 606, 706 may have a skin-contact adhesive layer disposed on its front face 506A, 606A, 706A to couple the transducer 500, 600, 700 to a subject's body. The skin-contact adhesive layer may comprise a biocompatible conductive adhesive.

The flexible layer 506, 606, 706 may be electrically non-conductive. For example, the flexible layer 506, 606, 706 may be made from a compressible material including but not limited to foam, rubber, elastomer, tape, bandage, or plaster. In some embodiments, the flexible layer 506, 606, 706 may be an adhesive coated foam. In some embodiments, the flexible layer 506, 606, 706 may be 3M™ Tegaderm™ Transparent Film Dressing Frame, which may include adhesive on one side facing the patient's body and facing away from the anisotropic material layer 504, 604, 704. Alternatively, as is explained in further detail above in reference to FIGS. 1A-2E, the flexible layer 506, 606, 706 may not include an adhesive coating such that the transducer 500, 600, 700 may instead include an adhesive layer such as electrically conductive skin contact adhesive layer 112, 212. Such adhesives may be as described herein.

As shown in FIGS. 5A-7B, the anisotropic material layer 504, 604, 704 may be a rounded V-shape or a boomerang shape. Alternatively, the anisotropic material layer 504, 604, 704 may be any other suitable shape including but not limited to a rectangular shape, a rounded rectangular shape, a triangular shape, a rounded triangular shape, a circular shape, an oval shape, a U-shape, a C-shape, a substantially rectangular shape, a substantially rounded rectangular shape, a substantially triangular shape, a substantially rounded triangular shape, a substantially circular shape, a substantially oval shape, a substantially U-shape, a substantially c-shape, a substantially rounded V-shape, or a substantially boomerang shape. The transducer array may independently be similarly shaped to the above shapes. Moreover, transducer array may be similarly shaped to the anisotropic material layer 504, 604, 704 constituent of the transducer array. In some embodiments, the flexible layer 506, 606, 706, may be coupled to the anisotropic material layer 504, 604, 704 by an adhesive layer disposed on the front face 504A, 604A, 704A of the anisotropic material layer 504, 604, 704 to couple the anisotropic material layer 504, 604, 704 to the flexible layer 506, 606, 706; this adhesive layer may or may not have an adhesive strength which is less than the adhesive strength of the adhesive layer disposed on the front face 506A, 606A, 706A of the flexible layer 506, 606, 706. Alternatively, the flexible layer 506, 606, 706 may adhere to the anisotropic material layer 504, 604, 704 via an adhesive layer disposed on the back face of the flexible layer. As will be explained in further detail below, the flexible layer 506, 606, 706 may be disposed about a perimeter 508, 608, 708 of the anisotropic material layer 504, 604, 704; in this way, the flexible layer 506, 606, 706 may have a perimeter portion outline shape which is substantially similar to that of the anisotropic material layer 504, 604, 704 and which may partially cover the interior portion 510, 610, 710 of the anisotropic material layer 504, 604, 704. In some embodiments, the interior portion 510, 610, 710 of the anisotropic material layer 504, 604, 704 includes at least one area that is at least 0.5 cm, or in some embodiments at least 1 cm, inside the perimeter of the anisotropic material layer 504, 604, 704. In some embodiments, the flexible layer 506, 606, 706 may cover at least 1% and at most 40% of the surface area of the anisotropic material layer 504, 604, 704 when viewed from its front face 504A, 604A, 704A.

As illustrated in FIGS. 5A, 5C, 6, and 7A-B, the flexible layer 506, 606, 706 may have a similar overlapping arrangement with respect to the front face 504A, 604A, 704A and to the perimeter of the anisotropic material layer 508, 608, 708 of the anisotropic material layer 504, 604, 704. As discussed above, when viewed in cross-section, as in FIGS. 1B-1D, 1F, 1H, 2B, and 2D, the flexible layer 108, 108A, 208, 208A may have a different shape. For example, in FIGS. 1B and 2B, the flexible layer 108, 208 may have an “L” shape. For example, in FIGS. 1C and 1D, the flexible layer 108 may have a “C” shape. For example, in FIGS. 1F, 1G, and 2D, the flexible layer 108A, 208A may have an S″ shape or a “Z” shape. For each of these constructs, a portion of the flexible layer 108, 108A, 208, 208A, 506, 606, 706 functions as an edge seal around the edge 604G (FIG. 6) of the anisotropic material layer 504, 604, 704. Additionally, as shown in FIG. 1H, a portion of flexible layer 108 between the anisotropic material layer 106 and the patient may have an angled profile relative to the anisotropic material layer 106.

As shown in FIGS. 5A, 5C, and FIG. 6, when viewed from the front face 504A, 604A, 704A of anisotropic material layer 504, 604, 704, the anisotropic material layer 504, 604, 704 may include one or more slits 516A-E, 616A-E or other cutout features to improve the flexibility of the transducer 500, 600, 700. In some embodiments, the slits 516A-E, 616A-E may extend from the perimeter 508, 608, 708 of the anisotropic material layer 504, 604, 704 towards the interior portion 510, 610, 710 of the anisotropic material layer 504, 604, 704. In FIG. 5C, slit 516C is not labeled because the slit is obscured from view by transducer wire 520. The flexible layer 506, 606, 706 may include at least one radial portion 512A-E extending towards the interior 510, 610, 710 of the anisotropic material layer 504, 604, 704. In some embodiments, the at least one radial portion 512A-E may extend along the length of, and cover, the slits 516A-E, 616A-E such that slits are not visible when viewed from the front face 504A, 604A, 704A of anisotropic material layer 504, 604, 704. Furthermore, radial portions 512A-E may have a single thickness along their entire length. Alternatively, radial portions 512A-E may have a first portion having a first thickness, a second portion having a second thickness, and so on. This feature is further illustrated in FIG. 6, with respect to first radial portions 626A-C and second radial portions 628A-C. The radial portions 512A-E may extend only partially towards the interior portion 510 of the anisotropic material layer 504, 604, 704 (see, example, 512D and 512E in FIG. 5A), or may extend from a first point along the perimeter 514, 614, 714 of the flexible layer 506, 606, 706 to a second point along the perimeter 514, 614, 714 (see, example, 512A, 512B, and 512C in FIG. 5A). In one example shown in FIG. 5A, a first portion of the respective radial portions 512A-E may extend from a first point along the perimeter 514, 614, 714 to an endpoint of the slits 516A-E, 616A-E, and a second portion of the respective radial portions 512A-E may extend from the endpoint of the slits 516A-E, 616A-E to the second point along the perimeter 514, 614, 714 such that the first portion has a first thickness and the second portion has a second thickness which is less than the first thickness (see, example, 512A, 512B, and 512C in FIG. 5A).

Similar to the hole 322 and the hole covering portion 342 of FIGS. 3A-3C, some embodiments may include a hole passing through both the front face 504A, 604A, 704A and the back face 504B, 704B of the anisotropic material layer 504, 604, 704, wherein, when viewed from the front face of the anisotropic material layer 504, 604, 704, the flexible layer comprising adhesive 506, 606, 706 may include a hole-covering portion to cover the hole on the front face of the anisotropic material layer.

As can be seen in FIGS. 5A, 6, and 7A, the flexible layer comprising adhesive 506, 606, 706 may divide the area of the front face 504A, 604A, 704A of the anisotropic material layer 504, 604, 704 into a plurality of sections 504C-F when viewed from the front face 504A, 604A, 704A. Some embodiments may include three, four, and any other suitable number of sections 504C-F. The individual sections 504C-F may have any suitable surface area including at least 1.0 cm² and at most 95.0 cm², and/or cover at least 5% and at most 45% of the surface area of the front face 504A, 604A, 704A. Relatedly, the sections 504C-F may have a combined surface area of at least 50.0 cm² and at most 400.0 cm², and/or cover at least 5% and at most 95% of the surface area of the front face 504A, 604A, 704A. In this way, a ratio of the surface area of the flexible layer comprising adhesive 506, 606, 706 to the combined surface area of the sections 504C-F may be calculated. In some embodiments, this ratio may be at least 0.1 and at most 0.5, however those skilled in the art will also understand that other ratios may also be possible.

The sections 504C-F may have any number of suitable sizes, shapes, and/or configurations. In one exemplary embodiment, the sections 504C-F may all have the same size and/or shape; in another embodiment, some of the sections 504C-F may have different sizes or different shapes, but not both; in yet another embodiment, all the sections 504C-F may have both different sizes and different shapes. In the case of the former two of these embodiments, the anisotropic material layer 504, 604, 704 may include at least one section 504C-F which may be a mirror image of another section 504C-F.

As explained previously with reference to FIGS. 1A-2E, the transducer 500, 600, 700 may also include adhesive layer 112, 212; in this way, each of the sections 504C-F may include an adhesive layer 112, 212 disposed thereon. In some embodiments, the adhesive layer 112, 212 may have a lower peel strength or tack strength than the adhesive of the flexible layer 506, 606, 706.

As shown in FIGS. 5B-6 and 7B, transducer 500, 600, 700 may include a transducer wire 520, 620, 720 and a connector 518, 618, 718 disposed on the array of electrode elements 502, 602, 702 for connecting the transducer 500, 600, 700 to transducer wire 520, 620, 720. In some embodiments, the transducer 500, 600, 700 may include a connector cover 722 (as depicted in FIG. 7B) disposed on the back face 518A, 718A of connector 518, 618, 718. Some embodiments may also include a connector cover 724 (e.g., as depicted in FIG. 7A), which may be part of or separate from the flexible layer comprising adhesive 706. In this way, connector cover 724 may be aligned with and opposite connector cover 722 (e.g., as depicted in FIGS. 7A and 7B). The connector covers 722, 724 may be foam, rubber, elastomer, tape, bandage, epoxy, plaster, or any other suitable material. As such, first connector cover 722 and/or second connector cover 724 may provide comfort to the subject by cushioning interaction of the connector 518, 618, 718 with the subject and/or the subject's garments.

As shown in FIG. 6, the transducer 600 may include the substrate 632, which may assist in holding together various components of the transducer 600 The transducer 600 may be affixed to the subject's body with the aid of the substrate 632. Suitable materials for the substrate 632 may include, for example, cloth, foam, flexible plastic, and/or a conductive medical gel or adhesive. The substrate 632 may take the form of an adhesive bandage (e.g., a medical bandage).

As is also shown in FIG. 6, the transducer 600 may be packaged prior to use with peel-off covers 630A-B disposed on the front face 606A of the flexible layer 606.

As explained above with reference to FIGS. 4A-4E, the transducer 500, 600, 700 may comprise two transducer subassemblies connected with a flexible electrical connector. Although references herein to FIGS. 5A-7B assume a single transducer assembly, one having skill in the art will appreciate that the teachings of FIGS. 4A-4E and associated disclosures will apply equally to the embodiments shown in FIGS. 5A-7B.

### Illustrative Embodiments

The invention may include other illustrative embodiments (“Embodiments”) as follows.

Embodiment 1. A transducer apparatus for delivering tumor treating fields to a subject's body, the transducer apparatus comprising: an array of one or more electrode elements, the array configured to be positioned over the subject's body with a front face of the array facing the subject's body; an anisotropic material layer electrically coupled to the array and located on a front side of the front face of the array, the anisotropic material layer comprising a front face and a back face, the back face facing the array; and a flexible layer coupled to the front face of the anisotropic material layer and configured to contact the subject's body; wherein the flexible layer comprises: a flexible material other than an adhesive; and, optionally, an adhesive.

Embodiment 2. The transducer apparatus of Embodiment 1, wherein the flexible layer comprises a layer of foam having adhesive disposed at least on a front face of the foam when viewed from a direction perpendicular to the front face of the anisotropic material layer.

Embodiment 3. The transducer apparatus of Embodiment 1 or Embodiment 2, wherein when viewed from the direction perpendicular to the front face of the anisotropic material layer, the flexible layer is disposed about a perimeter of the anisotropic material layer.

Embodiment 4. The transducer apparatus of Embodiment 3, wherein when viewed from the direction perpendicular to the front face of the anisotropic material layer, the front face of the anisotropic material layer comprises an interior portion which is partially covered by the flexible layer.

Embodiment 4A. The transducer apparatus of Embodiment 4, wherein when viewed from the direction perpendicular to the front face of the anisotropic material layer, the flexible layer comprises at least one portion radiating inward from the perimeter of the anisotropic material layer.

Embodiment 5. The transducer apparatus of Embodiment 3, wherein the anisotropic material layer comprises an edge, and the flexible layer defines a seal around the edge of the anisotropic material layer.

Embodiment 6. The transducer apparatus of Embodiment 1 or Embodiment 2, wherein when viewed from the direction perpendicular to the front face of the anisotropic material layer, the front face of the anisotropic material layer comprises a perimeter and an interior portion; and when viewed from the direction perpendicular to the front face of the anisotropic material layer, the flexible layer comprises: a perimeter portion on the front face of the anisotropic material layer and adjacent the perimeter of the anisotropic material layer; and at least one radial portion extending from the perimeter portion of the flexible layer towards the interior portion of the anisotropic material layer.

Embodiment 7. The transducer apparatus of Embodiment 6, wherein when viewed from the direction perpendicular to the front face of the anisotropic material layer, the at least one radial portion extends from a first point on the perimeter portion of the flexible layer to a second point on the perimeter portion of the flexible layer.

Embodiment 8. The transducer apparatus of Embodiment 1 or Embodiment 2, wherein when viewed from the direction perpendicular to the front face of the anisotropic material layer, the flexible layer divides the area of the front face of the anisotropic material layer and defines a plurality of sections of the anisotropic material layer.

Embodiment 8A. The transducer apparatus of Embodiment 8, wherein when viewed from the direction perpendicular to the front face of the anisotropic material layer, the anisotropic material layer comprises three or four sections defined by the flexible layer.

Embodiment 8B. The transducer apparatus of Embodiment 8, wherein when viewed from the direction perpendicular to the front face of the anisotropic material layer, the plurality of sections of the anisotropic material layer have an adhesive layer having a lower tack or peel strength than the flexible layer.

Embodiment 8C. The transducer apparatus of Embodiment 8, wherein when viewed from the direction perpendicular to the front face of the anisotropic material layer, a ratio of a surface area of the flexible layer to a combined surface area of the plurality of sections is at least 0.1 to at most 0.5.

Embodiment 8D. The transducer apparatus of Embodiment 8, wherein when viewed from the direction perpendicular to the front face of the anisotropic material layer, the plurality of sections of the anisotropic material layer are at least one of a same size or a same shape.

Embodiment 8E. The transducer apparatus of Embodiment 8, wherein when viewed from the direction perpendicular to the front face of the anisotropic material layer, the plurality of sections of the anisotropic material layer comprise at least one section having at least one of a different size or a different shape than others of the plurality of sections.

Embodiment 8F. The transducer apparatus of Embodiment 8, wherein when viewed from the direction perpendicular to the front face of the anisotropic material layer, the anisotropic material layer includes at least one section of the plurality of sections being a mirror image of at least one other section of the plurality of sections.

Embodiment 8G. The transducer apparatus of Embodiment 8, wherein when viewed from the direction perpendicular to the front face of the anisotropic material layer, each of the plurality of sections of the anisotropic material layer comprises a surface area of at least 1.0 cm² and at most 95.0 cm².

Embodiment 8H. The transducer apparatus of Embodiment 8, wherein when viewed from the direction perpendicular to the front face of the anisotropic material layer, the plurality of sections of the anisotropic material layer comprises a combined surface area of at least 50.0 cm² and at most 400.0 cm².

Embodiment 8I. The transducer apparatus of Embodiment 8, wherein when viewed from the direction perpendicular to the front face of the anisotropic material layer, each of the plurality of sections of the anisotropic material layer covers a surface area of at least 5% and at most 45% of a surface area of the front face of the anisotropic material layer.

Embodiment 8J. The transducer apparatus of Embodiment 8, wherein when viewed from the direction perpendicular to the front face of the anisotropic material layer, the plurality of sections of the anisotropic material layer comprises a combined surface area which covers at least 30% and at most 95% of a surface area of the front face of the anisotropic material layer.

Embodiment 9. The transducer apparatus of Embodiment 2, wherein the front face of the anisotropic material layer comprises a perimeter and an interior portion, and the anisotropic material layer comprises at least one slit extending from the perimeter of the front face of the anisotropic material layer towards the interior portion of the front face of the anisotropic material layer, wherein when viewed from a direction perpendicular to the front face of the anisotropic material layer, a portion of the flexible layer extends along and covers the at least one slit.

Embodiment 10. The transducer apparatus of Embodiment 9, wherein when viewed from the direction perpendicular to the front face of the anisotropic material layer, the portion of the flexible layer further extends from a first point of the perimeter of the front face of the anisotropic material layer to a second point of the perimeter of the front face of the anisotropic material layer.

Embodiment 11. The transducer apparatus of Embodiment 10, wherein when viewed from the direction perpendicular to the front face of the anisotropic material layer, the portion of the flexible layer has a thicker portion covering the at least one slit and a thinner portion not covering the at least one slit.

Embodiment 11A. The transducer apparatus of Embodiment 11, wherein when viewed from the direction perpendicular to the front face of the anisotropic material layer, the flexible layer further comprises another portion disposed about a perimeter of the anisotropic material layer.

Embodiment 12. The transducer apparatus of Embodiment 1 or Embodiment 2, wherein the anisotropic material layer further comprises a hole passing through the front face and the back face of the anisotropic material layer, and when viewed from the direction perpendicular to the front face of the anisotropic material layer, the flexible layer comprises a hole-covering portion to cover the hole on the front face of the anisotropic material layer.

Embodiment 13. The transducer apparatus of Embodiment 1 or Embodiment 2, further comprising: a first transducer subassembly and a second transducer subassembly, each of the first and second transducer subassemblies comprising: a portion of the electrode array; a portion of the anisotropic material layer; and a portion of the flexible layer; and a flexible electrical connector configured to electrically connect the first transducer subassembly to the second transducer subassembly, the flexible electrical connector comprising: a front face facing the subject's body; a back face opposite the front face; and at least one connector flexible layer disposed on at least one of the front face and the back face; wherein the flexible layer comprises: a flexible material other than an adhesive; and, optionally, an adhesive.

Embodiment 13A. The transducer apparatus of Embodiment 13, wherein the connector flexible layer disposed on the front face is contiguous with the flexible layer.

Embodiment 13B. The transducer apparatus of Embodiment 13, wherein at least a portion of the flexible layer is disposed about a perimeter of the anisotropic material layer.

Embodiment 14. The transducer apparatus of Embodiment 1 or Embodiment 2, further comprising: a connector for electrically connecting a conductive wire to the array of one or more electrode elements, the connector comprising a back face facing away from the subject's body; and either (a) a first connector cover disposed on the back face of the connector; or a second connector cover aligned with and located opposite the connector; or both (a) and (b).

Embodiment 14A. The transducer apparatus of Embodiment 14, wherein the flexible layer comprises a second connector cover aligned with and located opposite the connector.

Embodiment 14B. The transducer apparatus of Embodiment 14A, wherein the flexible layer further comprises another portion disposed about a perimeter of the anisotropic material layer.

Embodiment 15. The transducer apparatus of Embodiment 1 or Embodiment 2, wherein when the flexible layer is viewed in cross section, the flexible layer comprises one of an L-shape, C-shape, Z-shape, and S-shape, or wherein when the flexible layer is viewed in cross section, the flexible layer has an angled profile.

Embodiment 15A. The transducer apparatus of Embodiment 1 or Embodiment 2, wherein the flexible layer defines a seal between the anisotropic material layer and the subject's body.

Embodiment 15B. The transducer apparatus of Embodiment 1 or Embodiment 2, wherein the flexible layer is foam.

Embodiment 15C. The transducer apparatus of Embodiment 1 or Embodiment 2, wherein the flexible layer is a compressible material.

Embodiment 15D. The transducer apparatus of Embodiment 1 or Embodiment 2, wherein the flexible layer is a rubber or elastomeric material.

Embodiment 15E. The transducer apparatus of Embodiment 1 or Embodiment 2, wherein the flexible layer is non-conductive.

Embodiment 15F. The transducer apparatus of Embodiment 1 or Embodiment 2, wherein a thickness of the flexible layer is at least 0.1 mm and at most 1.5 mm.

Embodiment 15G. The transducer apparatus of Embodiment 1 or Embodiment 2, wherein when viewed from the direction perpendicular to the front face of the anisotropic material layer, the flexible layer covers at least 1% and at most 40% of the anisotropic material layer.

Embodiment 15H. The transducer apparatus of Embodiment 1 or Embodiment 2, further comprising a skin-contact adhesive layer disposed on the front face of the anisotropic material layer.

Embodiment 16. The transducer apparatus of Embodiment 1 or Embodiment 2, wherein the flexible layer comprises a first adhesive having a first adhesive strength, and the front face of the anisotropic material layer comprises a second adhesive having a second adhesive strength less than the first adhesive strength.

Embodiment 17. The transducer apparatus of Embodiment 2, wherein the anisotropic material layer comprises graphite.

Embodiment 18. The transducer apparatus of Embodiment 1, wherein the flexible material of the flexible layer comprises a rubber or an elastomer.

Embodiment 18A. The transducer apparatus of Embodiment 1 or Embodiment 2, wherein when viewed from the direction perpendicular to the front face of the anisotropic material layer, the anisotropic material layer has a rectangular shape, a rounded rectangular shape, a triangular shape, a rounded triangular shape, a circular shape, an oval shape, a U-shape, a rounded V-shape, a boomerang shape, a substantially rectangular shape, a substantially rounded rectangular shape, a substantially triangular shape, a substantially rounded triangular shape, a substantially circular shape, a substantially oval shape, a substantially U-shape, a substantially rounded V-shape, or a substantially boomerang shape.

Embodiment 19. A transducer apparatus for delivering tumor treating fields to a subject's body, the transducer apparatus comprising: an array of one or more electrode elements, the array configured to be positioned over the subject's body with a front face of the array facing the subject's body; an anisotropic material layer electrically coupled to the array and located on a front side of the front face of the array, the anisotropic material layer comprising a front face and a back face, the back face facing the array; and a foam layer coupled to the front face of the anisotropic material layer, wherein when viewed from a direction perpendicular to the front face of the anisotropic material layer, at least a portion of the foam layer is disposed about a perimeter of the anisotropic material layer.

Embodiment 20. A transducer apparatus for delivering tumor treating fields to a subject's body, the transducer apparatus comprising: an array of one or more electrode elements, the array configured to be positioned over the subject's body with a front face of the array facing the subject's body; an anisotropic material layer electrically coupled to the array and located on a front side of the front face of the array, the anisotropic material layer comprising a front face and a back face, the back face facing the array; and a foam layer coupled to the front face of the anisotropic material layer, wherein the anisotropic material layer comprises at least one slit extending from a perimeter of the front face of the anisotropic material layer towards an interior portion of the front face of the anisotropic material layer or at least one hole extending from the front face to the back face of the anisotropic material layer, wherein when viewed from a direction perpendicular to the front face of the anisotropic material layer, the foam layer covers the at least one slit or hole of the anisotropic material layer.

Embodiment 21. A transducer apparatus for delivering tumor treating fields to a subject's body, the transducer apparatus comprising: an array of one or more electrode elements, the array configured to be positioned over the subject's body with a front face of the array facing the subject's body; an anisotropic material layer electrically coupled to the array and located on a front side of the front face of the array, the anisotropic material layer comprising a front face and a back face, the back face facing the array; and a flexible layer coupled to the front face of the anisotropic material layer and configured to contact the subject's body.

Although the invention has been described as several example transducers and various illustrated embodiments, features of the example transducers and the illustrated embodiments may be used interchangeably together unless otherwise indicated herein or otherwise clearly contradicted by context.

Optionally, for each embodiment described herein, the transducers may be capable of being supplied with an electrical signal having an alternating current waveform at frequencies in a range from about 50 kHz to about 1 MHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention need not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of one or more electrode elements, the array configured to be positioned over the subject's body with a front face of the array facing the subject's body;
an anisotropic material layer electrically coupled to the array and located on a front side of the front face of the array, the anisotropic material layer comprising a front face and a back face, the back face facing the array; and
a flexible layer coupled to the front face of the anisotropic material layer and configured to contact the subject's body;
wherein the flexible layer comprises:
(i) a flexible material other than an adhesive; and, optionally,
(ii) an adhesive.

2. The transducer apparatus of claim 1, wherein the flexible layer comprises a layer of foam having adhesive disposed at least on a front face of the foam when viewed from a direction perpendicular to the front face of the anisotropic material layer.

3. The transducer apparatus of claim 2, wherein when viewed from the direction perpendicular to the front face of the anisotropic material layer, the flexible layer is disposed about a perimeter of the anisotropic material layer.

4. The transducer apparatus of claim 3, wherein when viewed from the direction perpendicular to the front face of the anisotropic material layer, the front face of the anisotropic material layer comprises an interior portion which is partially covered by the flexible layer.

5. The transducer apparatus of claim 3, wherein the anisotropic material layer comprises an edge, and the flexible layer defines a seal around the edge of the anisotropic material layer.

6. The transducer apparatus of claim 2, wherein
when viewed from the direction perpendicular to the front face of the anisotropic material layer, the front face of the anisotropic material layer comprises a perimeter and an interior portion; and
when viewed from the direction perpendicular to the front face of the anisotropic material layer, the flexible layer comprises:
a perimeter portion on the front face of the anisotropic material layer and adjacent the perimeter of the anisotropic material layer; and
at least one radial portion extending from the perimeter portion of the flexible layer towards the interior portion of the anisotropic material layer.

7. The transducer apparatus of claim 6, wherein when viewed from the direction perpendicular to the front face of the anisotropic material layer, the at least one radial portion extends from a first point on the perimeter portion of the flexible layer to a second point on the perimeter portion of the flexible layer.

8. The transducer apparatus of claim 2, wherein when viewed from the direction perpendicular to the front face of the anisotropic material layer, the flexible layer divides the area of the front face of the anisotropic material layer and defines a plurality of sections of the anisotropic material layer.

9. The transducer apparatus of claim 2, wherein the front face of the anisotropic material layer comprises a perimeter and an interior portion, and the anisotropic material layer comprises at least one slit extending from the perimeter of the front face of the anisotropic material layer towards the interior portion of the front face of the anisotropic material layer,
wherein when viewed from a direction perpendicular to the front face of the anisotropic material layer, a portion of the flexible layer extends along and covers the at least one slit.

10. The transducer apparatus of claim 9, wherein when viewed from the direction perpendicular to the front face of the anisotropic material layer, the portion of the flexible layer further extends from a first point of the perimeter of the front face of the anisotropic material layer to a second point of the perimeter of the front face of the anisotropic material layer.

11. The transducer apparatus of claim 10, wherein when viewed from the direction perpendicular to the front face of the anisotropic material layer, the portion of the flexible layer has a thicker portion covering the at least one slit and a thinner portion not covering the at least one slit.

12. The transducer apparatus of claim 2, wherein
the anisotropic material layer further comprises a hole passing through the front face and the back face of the anisotropic material layer, and
when viewed from the direction perpendicular to the front face of the anisotropic material layer, the flexible layer comprises a hole-covering portion to cover the hole on the front face of the anisotropic material layer.

13. The transducer apparatus of claim 2, further comprising:
a first transducer subassembly and a second transducer subassembly, each of the first and second transducer subassemblies comprising:
a portion of the electrode array;
a portion of the anisotropic material layer; and
a portion of the flexible layer; and
a flexible electrical connector configured to electrically connect the first transducer subassembly to the second transducer subassembly, the flexible electrical connector comprising:
a front face facing the subject's body;
a back face opposite the front face; and
at least one connector flexible layer disposed on at least one of the front face and the back face;
wherein the flexible layer comprises:
(i) a flexible material other than an adhesive; and, optionally,
(ii) an adhesive.

14. The transducer apparatus of claim 2, further comprising:
a connector for electrically connecting a conductive wire to the array of one or more electrode elements, the connector comprising a back face facing away from the subject's body; and either:
(a) a first connector cover disposed on the back face of the connector; or
(b) a second connector cover aligned with and located opposite the connector; or
(c) both (a) and (b).

15. The transducer apparatus of claim 2, wherein when the flexible layer is viewed in cross section, the flexible layer comprises one of an L-shape, C-shape, Z-shape, and S-shape, or
wherein when the flexible layer is viewed in cross section, the flexible layer has an angled profile.

16. The transducer apparatus of claim 2, wherein
the flexible layer comprises a first adhesive having a first adhesive strength, and
the front face of the anisotropic material layer comprises a second adhesive having a second adhesive strength less than the first adhesive strength.

17. The transducer apparatus of claim 2, wherein the anisotropic material layer comprises graphite.

18. The transducer apparatus of claim 1, wherein the flexible material of the flexible layer comprises a rubber or an elastomer.

19. A transducer apparatus for delivering tumor treating fields to a subject's body, the transducer apparatus comprising:
an array of one or more electrode elements, the array configured to be positioned over the subject's body with a front face of the array facing the subject's body;
an anisotropic material layer electrically coupled to the array and located on a front side of the front face of the array, the anisotropic material layer comprising a front face and a back face, the back face facing the array; and
a foam layer coupled to the front face of the anisotropic material layer,
wherein when viewed from a direction perpendicular to the front face of the anisotropic material layer, at least a portion of the foam layer is disposed about a perimeter of the anisotropic material layer.

20. A transducer apparatus for delivering tumor treating fields to a subject's body, the transducer apparatus comprising:
an array of one or more electrode elements, the array configured to be positioned over the subject's body with a front face of the array facing the subject's body;
an anisotropic material layer electrically coupled to the array and located on a front side of the front face of the array, the anisotropic material layer comprising a front face and a back face, the back face facing the array; and
a foam layer coupled to the front face of the anisotropic material layer,
wherein the anisotropic material layer comprises at least one slit extending from a perimeter of the front face of the anisotropic material layer towards an interior portion of the front face of the anisotropic material layer or at least one hole extending from the front face to the back face of the anisotropic material layer,
wherein when viewed from a direction perpendicular to the front face of the anisotropic material layer, the foam layer covers the at least one slit or hole of the anisotropic material layer.
